(19) United States
(12) Patent Application Publication
(10) Pub. No.: US 2008/0171021 A1
(43) Pub. Date: Jul. 17, 2008

(54) PHARMACEUTICAL USE OF NITRIC OXIDE, HEME OXYGENASE-1 AND PRODUCTS OF HEME DEGRADATION

(75) Inventors: Fritz H. Bach, Manchester-by-the-sea, MA (US); Leo E. Otterbein, New Kensington, PA (US)

Correspondence Address: FISH & RICHARDSON PC, P.O. BOX 1022, MINNEAPOLIS, MN, 55440-1022, US

(73) Assignees: Beth Israel Deaconess Medical Center, Inc., Boston, MA (US); University of Pittsburgh of the Commonwealth System of Higher Education, Pittsburgh, PA (US)

(21) Appl. No.: 11/931,645

(22) Filed: Oct. 31, 2007

Related U.S. Application Data
(62) Division of application No. 10/600,182, filed on Jun. 20, 2003.
(60) Provisional application No. 60/390,457, filed on Jun. 21, 2002.

(51) Int. Cl. A01N 63/00; A61K 35/12

(52) U.S. Cl. 424/93.7; 424/572; 424/520

(57) ABSTRACT

The present invention relates to the treatment of disorders using nitric oxide (NO), heme oxygenase-1 (HO-1) and heme degradation products such as carbon monoxide (CO), biliverdin, bilirubin and iron.

### CROSS-REFERENCE TO RELATED APPLICATIONS

This application claims priority to U.S. Provisional Application No. 60/390,457, filed Jun. 21, 2002, which is incorporated herein by reference in its entirety.

### STATEMENT AS TO FEDERALLY SPONSORED RESEARCH

This invention was made with Government support under National Institutes of Health Grant No. HL 58688. The Government has certain rights in this invention.

### TECHNICAL FIELD

The present invention relates to the treatment of disorders using nitric oxide in combination with heme oxygenase-1 and/or heme degradation products, such as carbon monoxide.

### BACKGROUND

Nitric oxide (NO) is a highly reactive free radical compound produced by many cells of the body. It relaxes vascular smooth muscle by binding to the heme moiety of cytosolic guanylate cyclase, activating guanylate cyclase and increasing intracellular levels of cyclic guanosine 3′,5′-monophosphate (cGMP), leading to vasodilation.

Heme oxygenase-1 (HO-1) catalyzes the first step in the degradation of heme. HO-1 cleaves the α-meso carbon bridge of b-type heme molecules by oxidation to yield equimolar quantities of biliverdin IXa, carbon monoxide (CO), and free iron. Subsequently, biliverdin is converted to bilirubin via biliverdin reductase, and the free iron is sequestered into ferritin (the production of which is induced by the free iron).

### SUMMARY

The present invention is based, in part, on the discovery that the administration of NO in combination with the induction/expression/administration of HO-1 and/or the administration of other heme degradation products, e.g., CO, can be used to treat various disorders.

Accordingly, the present invention features a method of reducing inflammation in a patient. The method includes administering to a patient diagnosed as suffering from or at risk for inflammation: (i) a pharmaceutical composition comprising NO, and (ii) a second treatment selected from inducing HO-1 or ferritin in the patient using a suitable inducer other than NO, expressing HO-1 or ferritin in the patient, and administering a pharmaceutical composition comprising HO-1, CO, bilirubin, biliverdin, ferritin, iron, desferoxamine, salicylaldehyde isonicotinoyl hydrazone, iron dextran, or apoferritin, in amounts sufficient to reduce inflammation. The inflammation is preferably not associated with a hemoglobinopathy.

In one embodiment, the method includes administering both NO and a pharmaceutical composition that includes CO. The concentration of CO in the composition can fall within the range of about 0.0000001% to about 0.3% by weight, e.g., 0.0001% to about 0.25% by weight, preferably at least about 0.001%, e.g., at least about 0.005%, 0.010%, 0.02%, 0.025%, 0.03%, 0.04%, 0.05%, 0.06%, 0.08%, 0.10%, 0.15%, 0.20%, 0.22%, or 0.24% by weight of carbon monoxide. Preferred ranges of carbon monoxide include 0.001% to about 0.24%, about 0.005% to about 0.22%, about 0.01% to about 0.20%, and about 0.02% to about 0.1% by weight.

Another treatment of the invention involves administering both NO and a pharmaceutical composition that includes biliverdin. The pharmaceutical composition can be administered to the patient at a dosage of at least 1 micromole/kg/day of biliverdin, e.g., about 1 to 1000 micromoles/kg/day, e.g., 10 to 500 micromoles/kg/day, 20 to 200 micromoles/kg/day, or 25 to 100 micromoles/kg/day.

Alternatively or in addition, the treatment can include administering, in addition to NO, a pharmaceutical composition that includes bilirubin. The pharmaceutical composition can be administered to a patient to generate serum levels of bilirubin of at least about 1 μM, e.g., in a range of from about 1 to about 300 μM, e.g., about 10 to about 200 μM, or about 50 to about 100 μM. Individual doses of bilirubin can fall within the range of about 1 to 1000 mg/kg, e.g., 10 to 500 mg/kg, 20 to 200 mg/kg, or 25 to 150 mg/kg. The dosage will generally be at least 1 mg/kg.

Further, the treatment can include administering both NO and a pharmaceutical composition that includes apoferritin and/or ferritin to the patient. The apoferritin or ferritin can be administered to the patient at a dosage of at least 1 mg/kg, such as about 1 to 1000 mg/kg, e.g., 10 to 500 mg/kg, 20 to 200 mg/kg, and 25 to 150 mg/kg.

The treatment can also include administering both NO and a pharmaceutical composition that includes desferoxamine (DFO) to the patient. The DFO can be administered to the patient at a dosage of at least 0.1 mg/kg, such as about 0.1 to 1000 mg/kg, e.g., 0.5 to 800 mg/kg, 1 to 600 mg/kg, 2 to 400 mg/kg, or 2.5 to 250 mg/kg.

Further, the treatment can include administering both NO and a pharmaceutical composition that includes iron dextran to the patient. The iron dextran can be administered to the patient at a dosage of at least 1 mg/kg, such as about 1 to 1000 mg/kg, e.g., 10 to 900 mg/kg, 100 to 800 mg/kg, 300 to 700 mg/kg, or 400 to 600 mg/kg. Alternatively, free iron, e.g., in the form of iron supplements, can be delivered to the patient in molar equivalent doses.

The treatment can also include administering both NO and a pharmaceutical composition that includes salicylaldehyde isonicotinoyl hydrazone (SIH) to the patient. The SIH can be administered to the patient orally or parenterally at a dosage of at least 0.01 mmol/kg, such as about 0.02 to 100 mmol/kg, e.g., about 0.02 to 10 mmol/kg, e.g., 0.02 to 50 mmol/kg, or 0.2 to 20 mmol/kg.

The inflammation can be associated with a condition selected from the following group: asthma, adult respiratory distress syndrome, interstitial pulmonary fibrosis, pulmonary emboli, chronic obstructive pulmonary disease, primary pulmonary hypertension, chronic pulmonary emphysema, congestive heart failure, peripheral vascular disease, stroke, atherosclerosis, ischemia-reperfusion injury, heart attacks, glomerulonephritis, conditions involving inflammation of the kidney, infection of the genitourinary tract, viral and toxic hepatitis, cirrhosis, ileus, necrotizing enterocolitis, specific and non-specific inflammatory bowel disease, rheumatoid arthritis, deficient wound healing, Alzheimer's disease, Parkinson's disease, graft versus host disease, and hemorrhagic, septic, or anaphylactic shock.

In an embodiment of the present invention, the inflammation is inflammation of the heart, lung, liver, pancreas, joints, eye, bronchi, spleen, brain, skin, and/or kidney. The inflammation can also be an inflammatory condition localized in the gastrointestinal tract, e.g., amoebic dysentery, bacillary dysentery, schistosomiasis, campylobacter enterocolitis, yersinia enterocolitis, enterobius vermicularis, radiation enterocolitis, ischaemic colitis, eosinophilic gastroenteritis, ulcerative colitis, indeterminate colitis, and Crohn's disease. Alternatively, it can be a systemic inflammation.

In another aspect, the invention features a method of transplanting an organ, tissue, or cells, which includes administering to a donor (or to an organ of the donor in situ) a pharmaceutical composition comprising nitric oxide, in combination with administering at least one treatment selected from: inducing HO-1 or ferritin in the donor, expressing HO-1 or ferritin in the donor, and administering a pharmaceutical composition comprising CO, HO-1, bilirubin, biliverdin, ferritin, iron, DFO, SIH, iron dextran, or apoferritin to the donor, and transplanting an organ tissue or cells of the donor into a recipient, wherein the nitric oxide and treatment administered are sufficient to enhance survival or function of the transplant after transplantation into the recipient.

The invention also features a method of transplanting an organ, tissue, or cells, which includes (a) providing an organ, tissue, or cells of a donor; (b) administering to the organ, tissue, or cells ex vivo a pharmaceutical composition comprising nitric oxide, in combination with administering at least one treatment selected from: inducing HO-1 or ferritin in the organ, tissue, or cells, expressing HO-1 or ferritin in the organ, tissue, or cells, and administering a pharmaceutical composition comprising CO, HO-1, bilirubin, biliverdin, ferritin, DFO, SIH, iron dextran, or apoferritin; and (c) transplanting the organ, tissue, or cells into a recipient, wherein the nitric oxide and treatment administered to the organ are sufficient to enhance survival or function of the transplant after transplantation.

Further, the invention features a method of transplanting an organ, tissue, or cells, which includes providing an organ, tissue or cells from a donor, transplanting the organ, tissue or cells into a recipient, and before, during, or after step the transplanting step, administering to the recipient a pharmaceutical composition comprising nitric oxide, in combination with administering at least one treatment selected from: inducing HO-1 or ferritin in the recipient, expressing HO-1 or ferritin in the recipient, and administering a pharmaceutical composition comprising CO, HO-1, bilirubin, biliverdin, ferritin, DFO, SIH, iron dextran, or apoferritin; wherein the nitric oxide and treatment administered to the recipient are sufficient to enhance survival or function of the organ after transplantation of the organ to the recipient.

If desired, the NO part of this treatment can be administered at any one, two, or three of the following steps: (1) treatment of the donor prior to and/or during removal of the organ; (2) treatment of the organ ex vivo; and (3) treatment of the recipient prior to, during, or after transplant of the organ. The second treatment described herein (e.g., induction of HO-1, administration of CO, etc.) can be administered at the same time as, before, or after the NO. For example, both NO and CO could be administered to the donor, followed by bathing the organ in a biliverdin solution, followed by administration of NO and ferritin to the recipient. All other specific combinations and permutations of this method are contemplated, though not specifically listed herein.

The invention also provides a method of performing angioplasty on a patient, which includes performing angioplasty on the patient; and before, during, or after the performing step, administering to the patient a pharmaceutical composition comprising nitric oxide, in combination with administration of a second treatment selected from: inducing HO-1 or ferritin in the recipient, expressing HO-1 or ferritin in the patient, and administering a pharmaceutical composition comprising CO, HO-1, bilirubin, biliverdin, ferritin, DFO, SIH, iron dextran, or apoferritin. The nitric oxide and second treatment are administered in an amount sufficient to reduce (e.g., prevent) intimal hyperplasia in the patient. The angioplasty can be any angioplasty procedure, e.g., balloon angioplasty; laser angioplasty; atherectomy, e.g., directional atherectomy, rotational atherectomy, or extraction atherectomy; and/or any angioplasty procedure using a stent, or any combination of such procedures.

The invention also provides a method of treating (e.g., preventing or decreasing) restenosis or intimal hyperplasia in a patient. The method includes administering to a patient diagnosed as suffering from or at risk for restenosis: (i) a pharmaceutical composition comprising NO, and (ii) a second treatment selected from inducing HO-1 or ferritin in the patient using a suitable inducer other than NO, expressing HO-1 or ferritin in the patient, and administering a pharmaceutical composition comprising HO-1, CO, bilirubin, biliverdin, ferritin, iron, DFO, SIH, iron dextran, or apoferritin, in amounts sufficient to treat restenosis or intimal hyperplasia. The intimal hyperplasia or restenosis can be caused by balloon angioplasty; laser angioplasty; atherectomy, e.g., directional atherectomy, rotational atherectomy, or extraction atherectomy; and/or any angioplasty procedure using a stent, or any combination of such procedures.

The invention also features a method of performing surgery (e.g., other than transplant surgery) e.g., vascular and/or abdominal surgery, on a patient, which includes performing surgery on the patient; and before, during, or after performing the surgery, administering to the patient a pharmaceutical composition comprising nitric oxide, in combination with administering at least one treatment selected from: inducing HO-1 or ferritin in the recipient, expressing HO-1 or ferritin in the patient, and administering a pharmaceutical composition comprising CO, HO-1, bilirubin, biliverdin, ferritin, DFO, SIH, iron dextran, or apoferritin.

The invention features a method of treating a cellular proliferative and/or differentiative disorder (e.g., naturally arising cancer) in a patient, which includes identifying a patient suffering from or at risk for a cellular proliferative and/or differentiative disorder (e.g., naturally arising cancer); and administering to the patient a pharmaceutical composition comprising nitric oxide, in combination with administering at least one treatment selected from: inducing HO-1 or ferritin in the recipient, expressing HO-1 or ferritin in the patient, and administering a pharmaceutical composition comprising CO, HO-1, bilirubin, biliverdin, ferritin, DFO, SIH, iron dextran, or apoferritin to the patient, in amounts sufficient to treat the cellular proliferative and/or differentiative disorder.

Any type of cancer can be treated using the methods described herein. The cancer can be cancer found in any part(s) of the patent's body, e.g., cancer of the stomach, small intestine, colon, rectum, mouth/pharynx, esophagus, larynx, liver, pancreas, lung, breast, cervix uteri, corpus uteri, ovary, prostate, testis, bladder, skin, kidney, brain/central nervous system, head, neck, throat, bone, or any combination thereof. It can also be a hematopoietic disorder, such as leukemia.

For cancer treatment, the methods can be used alone or in combination with other methods for treating cancer in patients. Accordingly, in another embodiment, the methods described herein can include treating the patient using surgery (e.g., to remove a tumor or portion thereof), chemotherapy, immunotherapy, gene therapy, and/or radiation therapy. Treatments described herein can be administered to a patient at any point, e.g., before, during, and/or after the surgery, chemotherapy, immunotherapy, gene therapy, and/or radiation therapy.

In another aspect, the invention features a method of treating unwanted angiogenesis in a patient. The method includes administering to a patient diagnosed as suffering from or at risk for unwanted angiogenesis: (i) a pharmaceutical composition comprising NO, and (ii) a second treatment selected from inducing HO-1 or ferritin in the patient using a suitable inducer other than NO, expressing HO-1 or ferritin in the patient, and administering a pharmaceutical composition comprising HO-1, CO, bilirubin, biliverdin, ferritin, iron, DFO, SIH, iron dextran, or apoferritin, in amounts sufficient to treat unwanted angiogenesis.

The invention features a method of treating hepatitis in a patient. The method includes administering to a patient diagnosed as suffering from or at risk for hepatitis: (i) a pharmaceutical composition comprising NO, and (ii) a second treatment selected from inducing HO-1 or ferritin in the patient using a suitable inducer other than NO, expressing HO-1 or ferritin in the patient, and administering a pharmaceutical composition comprising HO-1, CO, bilirubin, biliverdin, ferritin, iron, DFO, SIH, iron dextran, or apoferritin, in amounts sufficient to treat hepatitis.

The hepatitis can be the result of, or a person may be considered at risk for hepatitis because of, any of a number of factors, e.g., infections, e.g., viral infections, e.g., infection with hepatitis A, B, C, D, E and/or G virus; alcohol use (e.g., alcoholism); drug use (e.g., one or more drugs described herein, e.g., acetaminophen, anesthetics, anti-tuberculosis drugs, antifungal agents, antidiabetic drugs, neuroleptic agents, and drugs used to treat HIV infection and AIDS); autoimmune conditions (e.g., autoimmune hepatitis); and/or surgical procedures.

In still another aspect, the invention features a method of reducing the effects of ischemia in a patient, which includes identifying a patient suffering from or at risk for ischemia; and administering to the patient a pharmaceutical composition comprising nitric oxide, in combination with administering at least one treatment selected from: inducing HO-1 or ferritin in the recipient, expressing HO-1 or ferritin in the patient, and administering a pharmaceutical composition comprising CO, HO-1, bilirubin, biliverdin, ferritin, DFO, SIH, iron dextran, or apoferritin to the patient, in amounts sufficient to reduce the effects of ischemia.

Pharmaceutical compositions used in any of the treatment methods described herein can be in gaseous, liquid, or solid form, and can be administered to the patient by any method known in the art for administering gases and liquids to patients, e.g., via inhalation, insufflation, infusion, injection, and/or ingestion. In one embodiment of the present invention, the pharmaceutical composition is in gaseous or liquid (e.g., in the form of a mist or spray) form, and is administered to the patient by inhalation. If in liquid or solid form, the pharmaceutical composition can also be administered to the patient orally. In another embodiment, the pharmaceutical composition is in gaseous, solid, and/or liquid form, and is administered topically to an organ of the patient. In yet another embodiment, the pharmaceutical composition is in gaseous, liquid, and/or solid form, and is administered directly to the abdominal cavity of the patient. The pharmaceutical composition can also be administered to the patient by an extracorporeal membrane gas exchange device or an artificial lung.

The present invention also includes a vessel containing pressurized, medical grade gas comprising CO, NO, and optionally N₂, wherein the tank is labeled for use in medicine or surgery. For example, the vessel can bear a label indicating that the gas can be used to reduce inflammation in a patient, to treat cancer in a patient, to treat hepatitis in a patient, to treat unwanted angiogenesis in a patient, to treat arteriosclerosis in a patient, or used in conjunction with an angioplasty or surgical (e.g., transplant) procedure in a patient. The CO gas can be present in the vessel at a concentration of at least about 0.001%, e.g., at least about 0.005%, 0.010%, 0.020%, 0.025%, 0.030%, 0.005%, 0.100%, 0.500%, 1.0%, 2.0%, 10%, 50%, or 90% CO, and the NO gas can be present in the admixture at a concentration of at least about 0.0001%, e.g., at least about 0.0005%, 0.001%, 0.002%, 0.005%, 0.020%, 0.040%, 0.050%, 0.100%, 0.500%, 1.0%, 2.0%, 10%, 50%, or 90% NO, and essentially no O₂.

Also within the invention is the use of NO along with CO, HO-1, bilirubin, biliverdin, ferritin, DFO, SIH, iron dextran, and/or apoferritin, in the manufacture of a medicament for treatment or prevention of a condition described herein. The medicament can be in any form described herein, e.g., a liquid, gaseous, or solid composition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF THE DRAWINGS

FIG. 1 is a picture of a Western blot illustrating that the livers of CO-treated mice displayed increased expression of HO-1 in both the presence and absence of TNF-α/D-Gal. CO=carbon monoxide; Air=room air; TNF=TNF-α/D-Gal; β-Actin=control protein. Blot is representative of 2 independent experiments.

FIG. 2 is a picture of a Western blot illustrating that the livers of CO-treated mice do not display increased expression of HO-1 in the presence or absence of TNF-α/D-Gal if iNOS is inhibited using L-NIL. CO=carbon monoxide; Air=room air; TNF=TNF-α/D-Gal; β-Actin=control protein; L-NIL=L-N6-(1-iminoethyl)-lysine-dihydrochloride (a selective inhibitor of iNOS). Blot is representative of 2 independent experiments.

FIG. 3 is a bar graph illustrating that CO-induced HO-1 is protective against TNF-α-induced liver damage in mice. ALT=serum alanine aminotransferase; Air=room air; TNF=TNF-α/D-Gal; Sn=tin protoporphyrin (an inhibitor of HO-1); VP=V-PYRRO (a nitric oxide donor). Results are expressed as mean ±SD of 8-10 mice/group. *p<0.05 versus CO/TNF/D-gal-treated mice.

FIG. 4 is a bar graph illustrating that induction of HO-1 is protective against TNF-α-induced liver injury independent of iNOS activity. ALT=serum alanine aminotransferase; Air=room air; TNF=TNF-α/D-Gal; L-NIL=L-N6-(1-iminoethyl)-lysine-dihydrochloride (a selective inhibitor of iNOS); CoPP=cobalt protoporphyrin (an inducer of HO-1); iNOS^{-/-}=iNOS deficient mice. Results are mean ±SD of 6-8 mice/group. *p<0.001 versus Air/TNF and L-NIL/TNF.

FIG. 5 is bar graph illustrating that HO-1 expression is required for CO-induced protection of mouse hepatocytes from TNF-α/ActD-induced cell death. Wild type (black bars)=hepatocytes isolated from wild type C57BL/6J mice; hmox-1^{-/-} (white bars)=hepatocytes isolated from HO-1 null mice; CO=carbon monoxide; Air=room air; TNF-α=TNF-α/ActD. *p<0.01 versus non-TNF-α/ActD treated cells and versus TNF-α/ActD-treated cells that were also treated with CO.

FIG. 6 is bar graph illustrating that HO-1 expression is required for NO-induced protection of mouse hepatocytes from TNF-α/ActD-induced cell death. Wild type (black bars)=hepatocytes isolated from wild type C57BL/6J mice; hmox-1^{-/-} (white bars)=hepatocytes isolated from HO-1 null mice; SNAP=s-nitroso-N-acetyl-penicillamine (an NO donor); Air=room air; TNF-α=TNF-α/ActD. *p<0.01 versus non-TNF-α/ActD treated cells and versus TNF-α/ActD-treated cells that were also treated with NO.

FIG. 7 is a picture of a Western blot illustrating that CO augments LPS-induced iNOS expression in the liver of rats. Air=room air; CO=carbon monoxide; and LPS=lipopolysaccharide.

FIG. 8 is a bar graph illustrating that CO can inhibit LPS-induced liver injury as assessed by increased serum alanine aminotransferase (ALT) levels. Rats were administered 50 mg/kg, LPS, i.v.±CO (250 ppm) and blood was taken 8 hours later for serum ALT determination. Air=room air; CO=carbon monoxide; and LPS=lipopolysaccharide. Data is mean ±SD of 4-6 rats/group.

### DETAILED DESCRIPTION

The term “pharmaceutical composition” is used throughout the specification to describe a gaseous, liquid, or solid composition containing an active ingredient, e.g., NO, CO, an NO- or CO-releasing compound, HO-1 or ferritin (or an inducer of HO-1 or ferritin), bilirubin, and/or biliverdin, that can be administered to a patient and/or an organ. The invention contemplates use of any two, three, four, five, six, seven or eight of these in combination or in sequence. The skilled practitioner will recognize which form of the pharmaceutical composition, e.g., gaseous, liquid, and/or solid, is preferred for a given application. Further, the skilled practitioner will recognize which active ingredient(s) should be included in the pharmaceutical composition for a given application.

The term “patient” is used throughout the specification to describe an animal, human or non-human, rodent or non-rodent, to whom treatment according to the methods of the present invention is provided. Veterinary applications are clearly contemplated by the present invention. The term includes but is not limited to birds, reptiles, amphibians, and mammals, e.g., humans, other primates, pigs, rodents such as mice and rats, rabbits, guinea pigs, hamsters, cows, horses, cats, dogs, sheep and goats. Preferred subjects are humans, farm animals, and domestic pets such as cats and dogs. The term “treat(ment)” is used herein to describe delaying the onset of, inhibiting, or alleviating the effects of a disease or condition, e.g., a disease or condition described herein. Skilled practitioners will appreciate that a patient can be diagnosed by a physician (or veterinarian, as appropriate for the patient being diagnosed) as suffering from or at risk for a condition described herein by any method known in the art, e.g., by assessing a patient's medical history, performing diagnostic tests, and/or by employing imaging techniques. The compositions described herein can be administered (and/or administration can be supervised) by any person, e.g., a health-care professional, veterinarian, or caretaker (e.g., an animal (e.g., dog or cat) owner), depending upon the patient to be treated, and/or by the patient him/herself, if the patient is capable of self-administration.

The terms “effective amount” and “effective to treat,” as used herein, refer to an amount or concentration of active ingredients (e.g., NO and at least one of: CO, HO-1, ferritin (or an inducer of HO-1 or ferritin), bilirubin, and biliverdin) utilized for a period of time (including acute or chronic administration and periodic or continuous administration) that is effective within the context of its administration for causing an intended effect or physiological outcome. For example, an effective amount of a gaseous composition comprising NO and CO is an amount capable of reducing inflammation.

### Use of Nitric Oxide

The present invention includes providing NO to a patient, in conjunction with the administration of HO-1 and/or any or all of the products of heme degradation, e.g., CO, biliverdin, bilirubin, iron, and ferritin, to treat various diseases or conditions, and/or to improve the outcome of various surgical procedures. The term “nitric oxide” (or “NO”) as used herein describes molecular nitric oxide in its gaseous state, compressed into liquid form, or dissolved in aqueous solution. Pharmaceutical compositions comprising gaseous NO are typically administered by inhalation through the mouth or nasal passages to the lungs, where the NO may exert its effect directly or be readily absorbed into the patient's bloodstream. Compressed or pressurized gas, e.g., NO (and/or CO, as described in further detail below) useful in the methods of the invention can be obtained from any commercial source, and in any type of vessel appropriate for storing compressed gas. For example, compressed or pressurized gases can be obtained from any source that supplies compressed gases, such as oxygen, for medical use.

NO for inhalation is available commercially (e.g., INOmax™, INO Therapeutics, Inc., Clinton, N.J.). The gas may be obtained from commercial supplier typically as a mixture of 200-800 ppm NO in pure N₂ gas. The source of NO can be essentially 100% NO, or diluted with N₂ or any other inert gas (e.g., helium) to any desired concentration. It is vital that the NO be obtained and stored as a mixture free of any contaminating O₂ or higher oxides of nitrogen, because such higher oxides of nitrogen (which can form by reaction of O₂ with NO) are potentially harmful to lung tissues. The NO-containing gas is mixed with an O₂ containing gas (such as air or pure O₂) immediately prior to inhalation, minimizing the time that the NO is in contact with O₂. This can readily be accomplished by continuous mixing of the NO with the O₂-containing gas so that the two are in contact less than 20 seconds (preferably less than 10 seconds). If desired, purity of the NO may be demonstrated with chemiluminescence analysis, using known methods, prior to administration to the patient. Chemiluminescence NO—NOₓ analyzers are commercially available (e.g., Model 14A, Thermo Environmental Instruments, Franklin, Mass.). The NO—N₂ mixture may be blended with air or O₂ through, for example, calibrated rotameters which have been validated previously with a spirometer. The final concentration of NO in the breathing mixture may be verified with a chemical or chemiluminescence technique well known to those in the field (e.g., Fontijin et al., Anal Chem 42:575 [1970]). Alternatively, NO and NO₂ concentrations may be monitored by means of an electrochemical analyzer. Any impurities such as NO₂ can be scrubbed by exposure to NaOH solutions, baralyme, or sodalime. As an additional control, the FiO₂ of the final gas mixture may also be assessed.

Pharmaceutical compositions comprising NO can be administered using any method in the art for administering gases to patients. Safe and effective methods for administration of NO by inhalation are described in, e.g., U.S. Pat. No. 5,570,683; U.S. Pat. No. 5,904,938; and Frostell et al., Circulation 83:2038-2047, 1991. Some exemplary methods for administering gases (such as CO) to patients are described in detail below, and can be used to administer NO. Examples of methods and devices that can be utilized to administer gaseous pharmaceutical compositions comprising NO to patients include ventilators, face masks and tents, portable inhalers, intravenous artificial lungs (see, e.g., Hattler et al., Artif. Organs 18(11):806-812, 1994; and Golob et al., ASAIO J., 47(5):432-437, 2001), and normobaric chambers. However, the properties of NO may allow/necessitate some modification of these methods. In a hospital or emergency field situation, administration of NO gas can be accomplished, for example, by attaching a tank of compressed NO gas in N₂, and a second tank of oxygen or an oxygen/N₂ mixture (such as air), to an inhaler designed to mix gas from two sources. By controlling the flow of gas from each source, the concentration of NO inhaled by the patient can be maintained at an optimal level. NO can also be mixed with room air, using a standard low-flow blender (e.g., Bird Blender, Palm Springs, Calif.). NO can be generated from N₂ and O₂ (i.e., air) by using an electric NO generator. A suitable NO generator is described in U.S. Pat. No. 5,396,882. In addition, NO can be provided intermittently from an inhaler equipped with a source of NO such as compressed NO or an electric NO generator. The use of an inhaler may be particularly advantageous if a second compound (e.g., a phosphodiesterase inhibitor as described in further detail below) is administered, orally or by inhalation, in conjunction with the NO.

Preferably, in an inhalable pharmaceutical composition comprising NO gas, the NO concentration at the time of inhalation is about 0.1 ppm to about 300 ppm, e.g., 0.5 ppm to 290 ppm, 1.0 ppm to 280 ppm, 5 ppm to 250 ppm, 10 ppm to 200 ppm, or 10 ppm to 100 ppm, in air, pure oxygen, or another suitable inhalable gas or gas mixture. A suitable starting dosage for NO administered by inhalation can be 20 ppm (see, e.g., INOmax™ package insert), and the dosage can vary, e.g., from 0.1 ppm to 100 ppm, depending on the age and condition of the patient, the disease or disorder being treated, and other factors that the treating physician may deem relevant. Acute, sub-acute, and chronic administration of NO is contemplated by the present invention. NO can be delivered to the patient for a time (including indefinitely) sufficient to treat the condition and exert the intended pharmacological or biological effect. The concentration can be temporarily increased for short periods of time, e.g., 5 min at 200 ppm NO. This can be done when an immediate effect is desired. Preferred periods of time for exposure of a patient to NO include at least one hour, e.g., at least six hours; at least one day; at least one week, two weeks, four weeks, six weeks, eight weeks, ten weeks or twelve weeks; at least one year; at least two years; and at least five years. The patient can be exposed to the atmosphere continuously or intermittently during such periods. The administration of pharmaceutical compositions comprising NO (and/or CO) can be via spontaneous or mechanical ventilation.

When inhaled NO is administered, it is desirable to monitor the effects of the NO inhalation. Such monitoring can be used in a particular individual to verify desirable effects and to identify undesirable side effects that might occur. Such monitoring is also useful in adjusting dose level, duration, and frequency of administration of inhaled NO in a given individual.

Gaseous NO can be dissolved in aqueous solution, and utilized in that form. For example, such a solution could be used to bathe an organ, tissue or cells ex vivo, or used to perfuse an organ or tissue in situ. The solution can contain other active agents, e.g., CO, HO-1, heme, biliverdin, and/or bilirubin.

Alternatively or in addition, a NO-releasing compound can be administered to the patient. Examples of suitable NO-releasing compounds include, e.g., S-nitrosothiols such as S-nitroso-N-acetylpenicillamine, S-nitrocysteine, nitroprusside, nitrosoguanidine, glyceryl trinitrate, azide; hydroxylamine, and any NONOate compound (e.g., diethylamine/NONO, diethylenetriamine/NONO, and methylaminohexylmethylamine/NONO. An NO-releasing compound can be provided in powder form or as a liquid (e.g., by mixing the compound with a biologically-compatible excipient). Any one, or a combination, of the following routes of administration can be used to administer the NO-releasing compound(s) to the patient: intravenous injection, intraarterial injection, transcutaneous delivery, oral delivery, and inhalation (e.g., of a gas, powder or liquid).

It may be desirable to prolong the beneficial effects of inhaled NO within the patient. In determining how to prolong the beneficial effects of inhaled NO, it is useful to consider that one of the in vivo effects of NO is activation of soluble guanylate cyclase, which stimulates production of cGMP. At least some of the beneficial effects of NO may result from its stimulation of cGMP biosynthesis. Accordingly, a phosphodiesterase inhibitor can be administered in conjunction with NO inhalation to inhibit the breakdown of cGMP by endogenous phosphodiesterases.

The phosphodiesterase inhibitor can be introduced into a patient by any suitable method, including via an oral, transmucosal, intravenous, intramuscular, subcutaneous or intraperitoneal route. Alternatively, the inhibitor can be inhaled by the patient. For inhalation, the phosphodiesterase inhibitor is advantageously formulated as a dry powder or an aerosolized or nebulized solution having a particle or droplet size of less than 10 μm for optimal deposition in the alveoli, and may optionally be inhaled in a gas containing NO.

A suitable phosphodiesterase inhibitor is Zaprinast™ (M&B 22948; 2-o-propoxyphenyl-8-azapurine-6-one; Rhone-Poulenc Rorer, Dagenham Essex, UK). Zaprinast™ selectively inhibits the hydrolysis of cGMP with minimal effects on the breakdown of adenosine cyclic-monophosphate in vascular smooth muscle cells (Trapani et al., J Pharmacol Exp Ther 258:269, 1991; Harris et al., J Pharmacol Exp Ther 249:394, 1989; Lugnier et al., Biochem Pharmacol 35:1743, 1986; Souness et al., Br J Pharmacol 98:725, 1989). When using Zaprinast™ according to this invention, the preferred routes of administration are intravenous or oral. The suitable dose range may be determined by one of ordinary skill in the art. A stock solution of Zaprinast™ may be prepared in 0.05 N NaOH. The stock can then be diluted with Ringer's lactate solution to the desired final Zaprinast™ concentration, immediately before use.

This invention can be practiced with other phosphodiesterase inhibitors. Various phosphodiesterase inhibitors are known in the art, including Viagra® (sildenafil citrate), dipyridamole and theophylline. A suitable route of administration and suitable dose range can be determined by one of ordinary skill in the art.

Administration of NO with phosphodiesterase inhibitors can be performed as follows. In this example, the NO is administered at 20 ppm in air for 45 min. At the start of the 45 min period, 1.0 mg of Zaprinast™ per kg body weight is administered by intravenous infusion over 4 min, followed by a continuous infusion of 0.004 mg/kg/min for the rest of the 45 min period. Alternatively, at the start of the 45 min period, 0.15 mg dipyridamole per kg body weight is administered by intravenous infusion over 4 min, followed by a continuous infusion of 0.004 mg/kg/min for the rest of the 45 min period. The Zaprinast™ M or dipyridamole is administered in a saline solution.

### Use of Heme Oxygenase-1 and Products of Heme Degradation

In conjunction with administration of NO, the present invention includes providing to a patient heme oxygenase-1 (HO-1) by administering exogenously-produced HO-1 protein to the patient, by inducing HO-1 expression in the patient, and/or by expressing an exogenously-introduced gene encoding HO-1 in the patient, to treat various diseases or conditions, and/or to improve the outcome of various surgical procedures, e.g., transplantation procedures. Optionally, HO-1 can be provided to a patient in conjunction with administration of NO along with any or all of the products of heme degradation, e.g., carbon monoxide (CO), biliverdin, bilirubin, iron, and ferritin. Alternatively, any or all of the products of heme degradation can be provided to the patient, along with NO, without providing HO-1 to the patient.

Heme Oxygenase-1

HO-1 can be provided to a patient by inducing or expressing HO-1 in the patient, or by administering exogenous HO-1 directly to the patient. As used herein, the term “induce(d)” means to cause increased production of a protein, e.g., HO-1 or ferritin, in the body of a patient, using the patient's own endogenous (e.g., non-recombinant) gene that encodes the protein.

HO-1 can be induced in a patient by any method known in the art, preferably using an HO-1-inducing substance other than NO. For example, production of HO-1 can be induced by hemin, by iron protoporphyrin, or by cobalt protoporphyrin. A variety of non-heme agents including heavy metals, cytokines, hormones, COCl₂, endotoxin and heat shock are also strong inducers of HO-1 expression (Otterbein et al., Am. J. Physiol. Lung Cell Mol. Physiol. 279:L1029-L1037, 2000; Choi et al., Am. J. Respir. Cell Mol. Biol. 15:9-19, 1996; Maines, Annu. Rev. Pharmacol. Toxicol. 37:517-554, 1997; and Tenhunen et al., J. Lab. Clin. Med. 75:410-421, 1970). HO-1 is also highly induced by a variety of agents and conditions that create oxidative stress, including hydrogen peroxide, glutathione depletors, UV irradiation and hyperoxia (Choi et al., Am. J. Respir. Cell Mol. Biol. 15: 9-19, 1996; Maines, Annu. Rev. Pharmacol. Toxicol. 37:517-554, 1997; and Keyse et al, Proc. Natl. Acad. Sci. USA 86:99-103, 1989). A “pharmaceutical composition comprising an inducer of HO-1” means a pharmaceutical composition containing any agent capable of inducing HO-1 in a patient, e.g., any of the agents described above, e.g., hemin, iron protoporphyrin, and/or cobalt protoporphyrin.

The present invention contemplates that HO-1 (or ferritin) can be expressed in a patient via gene transfer. As used herein, the term “express(ed)” means to cause increased production of a protein, e.g., HO-1 or ferritin, in the body of a patient using an exogenously administered gene (e.g., a recombinant gene). The HO-1 or ferritin is preferably of the same species (e.g., human, mouse, rat, etc.) as the patient, in order to minimize any immune reaction. Expression could be driven by a constitutive promoter (e.g., cytomegalovirus promoters) or a tissue-specific promoter (e.g., milk whey promoter for mammary cells or albumin promoter for liver cells). An appropriate gene therapy vector (e.g., retroviruses, adenoviruses, adeno associated viruses (AAV), pox (e.g., vaccinia) viruses, human immunodeficiency virus (HIV), the minute virus of mice, hepatitis B virus, influenza virus, Herpes Simplex Virus-1, and lentiviruses) encoding HO-1 or ferritin would be administered to the patient orally, by inhalation, or by injection at a location appropriate for treatment of a condition described herein. Particularly preferred is local administration directly to the site of the condition. Similarly, plasmid vectors encoding HO-1 or ferritin can be administered, e.g., as naked DNA, in liposomes, or in microparticles.

Further, exogenous HO-1 protein can be directly administered to a patient by any method known in the art. Exogenous HO-1 can be directly administered in addition to, or as an alternative to the induction or expression of HO-1 in the patient as described above. The HO-1 protein can be delivered to a patient, for example, in liposomes, and/or as a fusion protein, e.g., as a TAT-fusion protein (see, e.g., Becker-Hapak et al., Methods 24, 247-256 (2001)). In the context of surgical procedures such as transplantation, it is contemplated that HO-1 can be induced and/or expressed in, and/or administered to donors, recipients, and/or the organ to be transplanted.

Heme Degradation Products

Additionally or alternatively, product(s) of heme degradation can be administered to patients to treat the diseases or conditions described herein. “Heme degradation products” include carbon monoxide, iron, biliverdin, bilirubin and (apo)ferritin. Any of the above can be provided to patients, e.g., as an active ingredient in a pharmaceutical composition or by other methods as described herein. Further, the present invention contemplates that iron-binding molecules other than ferritin, e.g., desferoxamine (DFO), iron dextran, and/or apoferritin, can be administered to the patient. Further still, the present invention contemplates that enzymes (e.g., biliverdin reductase) that catalyze the breakdown any of these products can be inhibited to create/enhance the desired effect. Any of the above can be administered, e.g., orally, intravenously, intraperitoneally, or topically.

Biliverdin and Bilirubin

The terms “biliverdin” and “bilirubin” refer to the linear tetrapyrrole compounds that are produced as a result of heme degradation.

Pharmaceutical compositions comprising biliverdin and/or bilirubin are typically administered to patients in aqueous or solid forms. Biliverdin and bilirubin useful in the methods of the invention can be obtained from any commercial source, e.g., any source that supplies chemicals for medical or laboratory use. In the preparation, use, or storage of biliverdin and bilirubin, it is recommended that the compounds be exposed to as little light as possible.

The amount of biliverdin and/or bilirubin to be included in pharmaceutical compositions and to be administered to patients will depend on absorption, distribution, inactivation, and excretion rates of the bilirubin and/or biliverdin, as well as other factors known to those of skill in the art. Effective amounts of biliverdin and/or bilirubin are amounts that are effective for treating a particular disease or condition.

Effective amounts of biliverdin can fall within the range of about 1 to 1000 micromoles/kg/day, e.g., at least 10 micromoles/kg/day, e.g., at least 20, 30, 40, 50, 60, 70, 80, 90, 100, 200, 300, 400, 500, 600, 700, 800, or 900 micromoles/kg/day. Preferred ranges include 10 to 500 micromoles/kg/day, 20 to 200 micromoles/kg/day, and 25 to 100 micromoles/kg/day. Because biliverdin is rapidly converted to bilirubin in the body (via biliverdin reductase), the present invention contemplates that doses of biliverdin above 1000 micromoles/kg/day can be administered to patients. The entire dose of biliverdin can be administered as a single dose, in multiple doses, e.g., several doses per day, or by constant infusion.

Effective amounts of bilirubin can be administered to a patient to generate serum levels of bilirubin in a range of from about 1 to about 300 μmols/L, e.g., about 10 to about 200 μmols/L, or about 50 to about 100 μmols/L. To generate such serum levels, individual doses of bilirubin can be administered, which can fall within the range of about 1 to 1000 mg/kg, e.g., 10, 20, 30, 40, 50, 60, 70, 80, 90, 100, 200, 300, 400, 500, 600, 700, 800, or 900 mg/kg. Preferred ranges include 10 to 500 mg/kg, 20 to 200 mg/kg, and 25 to 150 mg/kg. The entire dose of bilirubin can be administered as a single dose, in multiple doses, e.g., several doses per day, or by constant infusion.

A skilled practitioner will appreciate that amounts of bilirubin and/or biliverdin outside of these ranges may be used depending upon the application. Acute, sub-acute, and chronic administration of pharmaceutical compositions comprising biliverdin and/or bilirubin are contemplated by the present invention, depending upon, e.g., the severity or persistence of the disease or condition in the patient. The compositions can be delivered to the patient for a time (including indefinitely) sufficient to treat the condition and exert the intended pharmacological or biological effect.

The present invention contemplates that biliverdin and/or bilirubin can be bound to carriers. Such carriers include, for example, albumin or cyclodextrin. Binding of biliverdin and/or bilirubin to such a carriers could increase the solubility of biliverdin and/or bilirubin, thereby preventing deposition of biliverdin and/or bilirubin in the tissues. The present invention contemplates that it is possible to individually administer albumin along with unbound biliverdin and/or bilirubin and albumin to the patient to produce the desired effect.

Alternatively or in addition, it is contemplated that biliverdin reductase can be induced, expressed, and/or administered to a patient in situations where it is deemed desirable to increase bilirubin levels in the patient. The biliverdin reductase protein can be delivered to a patient, for example, in liposomes. Further, the present invention contemplates that increased levels of biliverdin reductase can be generated in a patient via gene transfer. An appropriate gene therapy vector (e.g., plasmid, adenovirus, adeno associated virus (AAV), lentivirus, or any of the other gene therapy vectors mentioned above) that encodes biliverdin reductase, with the coding sequence operably linked to an appropriate expression control sequence, would be administered to the patient orally, via inhalation, or by injection at a location appropriate for treatment of a condition described herein. In one embodiment of the present invention, a vector that encodes biliverdin reductase is administered to an organ affected by a condition described herein and biliverdin is subsequently or simultaneously administered to the organ, such that the biliverdin reductase breaks down the biliverdin to produce bilirubin in the organ.

Iron and Ferritin

The release of free iron by the action of HO-1 on heme stimulates the induction of apoferritin, which rapidly sequesters the iron to form ferritin. The present invention includes inducing or expressing ferritin in a patient to treat inflammation or ischemia or cell proliferation associated with various diseases or conditions in the patient. Ferritin can be induced in a patient by any method known in the art. For example, ferritin can be induced by administering iron dextran or free iron to the patient. As another example, ferritin levels in a patient can be increased by exposing the patient to ultraviolet radiation (Otterbein et al., Am. J. Physiol. Lung Cell Mol. Physiol. 279:L1029-L1037, 2000).

A “pharmaceutical composition comprising an inducer of ferritin” means a pharmaceutical composition containing any agent capable of inducing ferritin, e.g., heme, iron, and/or iron dextran, in a patient. Typically, a pharmaceutical composition comprising an inducer of ferritin is administered to a patient in aqueous or solid form. Inducers of ferritin, e.g., iron or iron dextran, useful in the methods of the invention can be obtained from any commercial source, e.g., a commercial source that supplies chemicals for medical or laboratory use.

An effective amount of an inducer of ferritin, e.g., iron or iron dextran, is an amount that is effective for treating a disease or condition. Effective doses of iron dextran can be administered once or several times per day, and each dose can fall within the range of about 1 to 1000 mg/kg, e.g., at least 2, 2.5, 5, 10, 20, 30, 40, 50, 60, 70, 80, 90, 100, 200, 250, 300, 400, 500, 600, 700, 800, or 900 mg/kg. Preferred ranges for iron dextran include 10 to 900 mg/kg, 100 to 800 mg/kg, 300 to 700 mg/kg, or 400 to 600 mg/kg. Free iron can be delivered to the patient, for example, as one or multiple doses of a commercially available iron supplement, e.g., a tablet containing iron.

Further, the present invention contemplates that increased levels of ferritin, e.g., H-chain ferritin, can be generated in a patient via gene transfer. An appropriate gene therapy vector (as described herein) would be administered to the patient orally or by injection or implantation at a location appropriate for treatment of a condition described herein. Further, exogenous ferritin can be directly administered to a patient by any method known in the art. Exogenous ferritin can be directly administered in addition to, or as an alternative to the induction or expression of apoferritin in the patient as described above. The ferritin protein can be delivered to a patient, for example, in liposomes, and/or as a fusion protein, e.g., as a TAT-fusion protein (see, e.g., Becker-Hapak et al., Methods 24:247-256, 2001).

Alternatively or in addition, it is contemplated that other iron-binding molecules can be administered to the patient to create or enhance the desired effect, e.g., to reduce free iron levels. For example, the present invention contemplates that apoferritin, as well as any type of iron chelator, e.g., desferioxamine (DFO) or salicylaldehyde isonicotinoyl hydrazone (SIH) (see, e.g., Blaha et al., Blood 91(11):4368-4372, 1998), can be administered to a patient to create or enhance the desired effect.

Effective doses of DFO can be administered once or several times per day, and each dose can fall within the range of from about 0.1 to 1000 mg/kg, e.g., at least about 2, 2.5, 5, 10, 20, 30, 40, 50, 60, 70, 80, 90, 100, 200, 250, 300, 400, 500, 600, 700, 800, or 900 mg/kg. Preferred ranges for DFO include 0.5 to 800 mg/kg, 1 to 600 mg/kg, 2 to 400 mg/kg, or 2.5 to 250 mg/kg.

Effective doses of SIH can be administered once or several times per day, and each dose can fall within the range of from about 0.02 to 100 mmol/kg, e.g., 0.02 to 50 mmol/kg, or 0.2 to 20 mmol/kg.

Effective doses of apoferritin can be administered once or several times per day, and each dose can fall within the range of about 1 to 1000 mg/kg, e.g., at least 2, 2.5, 5, 10, 20, 30, 40, 50, 60, 70, 80, 90, 100, 200, 250, 300, 400, 500, 600, 700, 800, or 900 mg/kg. Preferred ranges include 10 to 500 mg/kg, 20 to 200 mg/kg, and 25 to 150 mg/kg.

The skilled practitioner will recognize that any of the above, e.g., iron chelators, e.g., DFO or SIH, iron dextran, and apoferritin, can be administered as a single dose, in multiple doses, e.g., several doses per day, or by constant infusion. Further, any of the above can be administered continuously, and for as long as necessary to produce the desired effect. The skilled practitioner will recognize that any of the above can be administered in amounts outside the ranges given, depending upon the application.

Carbon Monoxide

The term “carbon monoxide” (or “CO”) as used herein describes molecular carbon monoxide in its gaseous state, compressed into liquid form, or dissolved in aqueous solution. An effective amount of carbon monoxide for use in the present invention is an amount that is effective for treating a disease or condition. For gases, effective amounts of carbon monoxide generally fall within the range of about 0.0000001% to about 0.3% by weight, e.g., 0.0001% to about 0.25% by weight, preferably at least about 0.001%, e.g., at least about 0.005%, 0.010%, 0.02%, 0.025%, 0.03%, 0.04%, 0.05%, 0.06%, 0.08%, 0.10%, 0.15%, 0.20%, 0.22%, or 0.24% by weight of carbon monoxide. Preferred ranges of carbon monoxide include, e.g., 0.002% to about 0.24%, about 0.005% to about 0.22%, about 0.01% to about 0.20%, and about 0.02% to about 0.1% by weight. For liquid solutions of CO, effective amounts generally fall within the range of about 0.0001 to about 0.0044 g CO/100 g liquid, e.g., at least about 0.0001, 0.0002, 0.0004, 0.0006, 0.0008, 0.0010, 0.0013, 0.0014, 0.0015, 0.0016, 0.0018, 0.0020, 0.0021, 0.0022, 0.0024, 0.0026, 0.0028, 0.0030, 0.0032, 0.0035, 0.0037, 0.0040, or 0.0042 g CO/100 g aqueous solution. Preferred ranges include, e.g., about 0.0010 to about 0.0030 g CO/100 g liquid, about 0.0015 to about 0.0026 g CO/100 g liquid, or about 0.0018 to about 0.0024 g CO/100 g liquid. A skilled practitioner will appreciate that amounts outside of these ranges may be used depending upon the application.

A carbon monoxide composition may be a gaseous carbon monoxide composition. Compressed or pressurized gas useful in the methods of the invention can be obtained from any commercial source, and in any type of vessel appropriate for storing compressed gas. For example, compressed or pressurized gases can be obtained from any source that supplies compressed gases, such as oxygen, for medical use. The term “medical grade” gas, as used herein, refers to gas suitable for administration to patients as defined herein. The pressurized gas including carbon monoxide used in the methods of the present invention can be provided such that all gases of the desired final composition (e.g., CO, He, NO, CO₂, O₂, N₂) are in the same vessel, except that NO and O₂ cannot be stored together. Optionally, the methods of the present invention can be performed using multiple vessels containing individual gases. For example, a single vessel can be provided that contains carbon monoxide, with or without other gases, the contents of which can be optionally mixed with the contents of other vessels, e.g., vessels containing oxygen, nitrogen, carbon dioxide, compressed air, or any other suitable gas or mixtures thereof.

Gaseous compositions administered to a patient according to the present invention typically contain 0% to about 79% by weight nitrogen, about 21% to about 100% by weight oxygen and about 0.0000001% to about 0.3% by weight (corresponding to about 1 ppb or 0.001 ppm to about 3,000 ppm) carbon monoxide. Preferably, the amount of nitrogen in the gaseous composition is about 79% by weight, the amount of oxygen is about 21% by weight and the amount of carbon monoxide is about 0.0001% to about 0.25% by weight. The amount of carbon monoxide is preferably at least about 0.001%, e.g., at least about 0.005%, 0.01%, 0.02%, 0.025%, 0.03%, 0.04%, 0.05%, 0.06%, 0.08%, 0.10%, 0.15%, 0.20%, 0.22%, or 0.24% by weight. Preferred ranges of carbon monoxide include 0.005% to about 0.24%, about 0.01% to about 0.22%, about 0.015% to about 0.20%, and about 0.025% to about 0.1% by weight. It is noted that gaseous carbon monoxide compositions having concentrations of carbon monoxide greater than 0.3% (such as 1% or greater) may be used for short periods (e.g., one or a few breaths), depending upon the application.

A gaseous carbon monoxide composition may be used to create an atmosphere that comprises carbon monoxide gas. An atmosphere that includes appropriate levels of carbon monoxide gas can be created, for example, by providing a vessel containing a pressurized gas comprising carbon monoxide gas, and releasing the pressurized gas from the vessel into a chamber or space to form an atmosphere that includes the carbon monoxide gas inside the chamber or space. Alternatively, the gases can be released into an apparatus that culminates in a breathing mask or breathing tube, thereby creating an atmosphere comprising carbon monoxide gas in the breathing mask or breathing tube, ensuring the patient is the only person in the room exposed to significant levels of carbon monoxide.

Carbon monoxide levels in an atmosphere can be measured or monitored using any method known in the art. Such methods include electrochemical detection, gas chromatography, radioisotope counting, infrared absorption, colorimetry, and electrochemical methods based on selective membranes (see, e.g., Sunderman et al., Clin. Chem. 28:2026-2032, 1982; Ingi et al., Neuron 16:835-842, 1996). Sub-parts per million carbon monoxide levels can be detected by, e.g., gas chromatography and radioisotope counting. Further, it is known in the art that carbon monoxide levels in the sub-ppm range can be measured in biological tissue by a midinfrared gas sensor (see, e.g., Morimoto et al., Am. J. Physiol. Heart. Circ. Physiol 280:H482-H488, 2001). Carbon monoxide sensors and gas detection devices are widely available from many commercial sources.

A pharmaceutical composition comprising carbon monoxide may also be a liquid composition. A liquid can be made into a pharmaceutical composition comprising carbon monoxide by any method known in the art for causing gases to become dissolved in liquids. For example, the liquid can be placed in a so-called “CO₂ incubator” and exposed to a continuous flow of carbon monoxide, preferably balanced with carbon dioxide, until a desired concentration of carbon monoxide is reached in the liquid. As another example, carbon monoxide gas can be “bubbled” directly into the liquid until the desired concentration of carbon monoxide in the liquid is reached. The amount of carbon monoxide that can be dissolved in a given aqueous solution increases with decreasing temperature. As still another example, an appropriate liquid may be passed through tubing that allows gas diffusion, where the tubing runs through an atmosphere comprising carbon monoxide (e.g., utilizing a device such as an extracorporeal membrane oxygenator). The carbon monoxide diffuses into the liquid to create a liquid carbon monoxide composition.

It is likely that such a liquid composition intended to be introduced into a living animal will be at or about 37° C. at the time it is introduced into the animal.

The liquid can be any liquid known to those of skill in the art to be suitable for administration to patients (see, for example, Oxford Textbook of Surgery, Morris and Malt, Eds., Oxford University Press (1994)). In general, the liquid will be an aqueous solution. Examples of solutions include Phosphate Buffered Saline (PBS), Celsior™, Perfadex™, Collins solution, citrate solution, and University of Wisconsin (UW) solution (Oxford Textbook of Surgery, Morris and Malt, Eds., Oxford University Press (1994)). In one embodiment of the present invention, the liquid is Ringer's Solution, e.g., lactated Ringer's Solution, or any other liquid that can be used infused into a patient. In another embodiment, the liquid includes blood, e.g., whole blood. The blood can be completely or partially saturated with carbon monoxide.

Any suitable liquid can be saturated to a set concentration of carbon monoxide via gas diffusers. Alternatively, pre-made solutions that have been quality controlled to contain set levels of carbon monoxide can be used. Accurate control of dose can be achieved via measurements with a gas permeable, liquid impermeable membrane connected to a carbon monoxide analyzer. Solutions can be saturated to desired effective concentrations and maintained at these levels.

A patient can be treated with a carbon monoxide composition, in conjunction with NO therapy, by any method known in the art of administering gases and/or liquids to patients. Carbon monoxide compositions can be prescribed for and/or administered to a patient diagnosed with, or determined to be at risk for any disease or condition described herein. The present invention contemplates the systemic administration of liquid or gaseous carbon monoxide compositions to patients (e.g., by inhalation and/or ingestion), and the topical administration of the compositions to the patient's organs, e.g., the gastrointestinal tract.

Gaseous carbon monoxide compositions are typically administered by inhalation through the mouth or nasal passages to the lungs, where the carbon monoxide may exert its effect directly or be readily absorbed into the patient's bloodstream. The concentration of active compound(s) (e.g., CO with or without NO) utilized in the therapeutic gaseous composition will depend on absorption, distribution, inactivation, and excretion (generally, through respiration) rates of the carbon monoxide as well as other factors known to those of skill in the art. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed invention. Treatments can be monitored and CO dosages can be adjusted to ensure optimal treatment of the patient. Acute, sub-acute and chronic administrations of carbon monoxide are contemplated by the present invention, depending upon, e.g., the severity or persistence of disease or condition in the patient. Carbon monoxide can be delivered to the patient for a time (including indefinitely) sufficient to treat the condition and exert the intended pharmacological or biological effect.

Examples of methods and devices that can be utilized to administer gaseous pharmaceutical compositions comprising carbon monoxide (and/or nitric oxide) to patients include ventilators, face masks and tents, portable inhalers, intravenous artificial lungs (see, e.g., Hattler et al., Artif. Organs 18(11):806-812, 1994; and Golob et al., ASAIO J., 47(5):432-437, 2001), and normobaric chambers, as described in further detail below.

The present invention further contemplates that aqueous solutions comprising carbon monoxide can be created for systemic delivery to a patient, e.g., by oral delivery to a patient.

Alternatively or in addition, carbon monoxide compositions can be applied directly to an organ or tissue of a patient. For example, carbon monoxide compositions can be applied to the interior and/or exterior of the entire gastrointestinal tract, or to any portion thereof, by any method known in the art for insufflating gases into a patient. Gases, e.g., carbon dioxide, are often insufflated into the gastrointestinal tract and the abdominal cavity of patients to facilitate examination during endoscopic and laparoscopic procedures, respectively (see, e.g., Oxford Textbook of Surgery, Morris and Malt, Eds., Oxford University Press (1994)). The skilled practitioner will appreciate that similar procedures could be used to administer carbon monoxide compositions directly to the gastrointestinal tract of a patient. The skin can be treated topically with a gaseous composition by, for example, exposing the affected skin to the gaseous composition in a normobarometric chamber (described herein), and/or by blowing the gaseous composition directly onto the skin. If the patient does not inhale the gas, the concentration of CO (and/or NO) in the gaseous composition could be as high as desired, e.g., over 0.25% and up to about 100%.

Liquid carbon monoxide compositions can also be administered directly to an organ or tissue of a patient. Liquid forms of the compositions can be administered by any method known in the art for administering liquids to patients. For example, the liquid compositions can be administered orally, e.g., by causing the patient to ingest an encapsulated or unencapsulated dose of the liquid carbon monoxide composition. As another example, liquids, e.g., saline solutions containing dissolved CO, can be injected into the gastrointestinal tract and the abdominal cavity of patients during endoscopic and laparoscopic procedures, respectively. The skilled practitioner will appreciate that similar procedures could be used to administer liquid compositions directly to an organ or tissue of a patient. Alternatively or in addition, in situ exposures or organs can be performed by any method known in the art, e.g., by in situ flushing of the organ with a liquid carbon monoxide composition during surgery (see Oxford Textbook of Surgery, Morris and Malt, Eds., Oxford University Press (1994)). The skin can be treated topically with a liquid composition by, for example, injecting the liquid composition into the skin. As a further example, the skin can be treated topically by applying the liquid composition directly to the surface of the skin, e.g., by pouring or spraying the liquid onto the skin and/or by submerging the skin in the liquid composition. Other externally-accessible surfaces such as the eye, mouth, throat, vagina, cervix, urinary tract, colon, and anus can be similarly treated topically with the liquid compositions.

The present invention also contemplates that compounds that release CO into the body after administration of the compound (e.g., CO-releasing compounds, e.g., photoactivatable CO-releasing compounds), e.g., dimanganese decacarbonyl, tricarbonyldichlororuthenium (II) dimer, and methylene chloride (e.g., at a dose of between 400 to 600 mg/kg, e.g., about 500 mg/kg), can also be used in the methods of the present invention, as can carboxyhemoglobin and CO-donating hemoglobin substitutes. Agents capable of delivering doses of CO (and/or NO) gas or liquid can also be utilized (e.g., CO releasing gums, creams, lozenges, ointments or patches).

Combination Therapy

The present invention contemplates that any of the treatments described above, e.g., the administration of NO, the induction/expression/administration of HO-1 and/or ferritin, and the administration of CO, bilirubin, and/or biliverdin, can be used individually or in any combination in surgical procedures and to treat the disorders or conditions described herein. Further, the present invention contemplates that in any treatment regimen using any combination of the above treatments, the treatments may be administered simultaneously on a single or multiple occasions, and/or individually at varying points in time, e.g., at different phases of a disease or condition. For example, a patient can receive CO and NO, both of those plus biliverdin, or NO plus bilirubin and ferritin, or NO plus two or more inducers of HO-1.

In particular, the present invention contemplates that both NO and CO can be administered to a patient. With regard to treatment protocols, NO and CO can be administered to the patient in any order and at any doses described herein. For example, a patient can be treated with NO prior to treatment with CO. In such instances, a patient can be exposed to at least one or multiple doses of NO, or exposed continuously to NO, beginning at a time ranging from about 1 minute to several days (e.g., about 1 hour, 2 hours, 5 hours, 12 hours, 1 day, 2 days or 3 days) before being exposed to CO. Alternatively, a patient can be treated with CO prior to treatment with NO, in a manner similar to that described above for treatment of a patient with NO prior to treatment with CO. Alternatively or in addition, a patient can be treated with NO and CO simultaneously, e.g., in a single exposure, multiple exposures, or during a continuous exposure. Alternatively or in addition, a patient can be exposed to NO and CO in an alternating manner. For example, a patient can be exposed first to NO, then to CO, then to NO, etc. Simultaneous exposures to NO and CO can optionally be included in alternating exposures.

In conjunction with NO therapy, amounts of CO effective to treat a disorder or condition described herein can be administered to (or prescribed for) a patient, e.g., by a physician or veterinarian, on the day the patient is diagnosed as suffering any of these disorders or conditions, or as having any risk factor associated with an increased likelihood that the patient will develop such disorder(s) or condition(s). Patients can inhale CO at concentrations ranging from 10 ppm to 1000 ppm, e.g., about 100 ppm to about 800 ppm, about 150 ppm to about 600 ppm, or about 200 ppm to about 500 ppm. Preferred concentrations include, e.g., about 30 ppm, 50 ppm, 75 ppm, 100 ppm, 125 ppm, 200 ppm, 250 ppm, 500 ppm, 750 ppm, or about 1000 ppm. CO can be administered to the patient intermittently or continuously. CO can be administered for at least about 1, 2, 4, 6, 8, 10, 12, 14, 18, or 20 days, or greater than 20 days, e.g., 1 2, 3, 5, or 6 months, or until the patient no longer exhibits symptoms of the condition or disorder, or until the patient is diagnosed as no longer being at risk for the condition or disorder. In a given day, CO can be administered continuously for the entire day, or intermittently, e.g., a single whiff of CO per day (where a high concentration is used), or for up to 23 hours per day, e.g., up to 20, 15, 12, 10, 6, 3, or 2 hours per day, or up to 1 hour per day.

With regard to surgical procedures, including transplantation procedures, CO can be administered systemically or locally to a patient prior to, during, and/or after a surgical procedure is performed, in conjunction with administration of NO therapy. Patients can inhale CO at concentrations ranging from 10 ppm to 1000 ppm, e.g., about 100 ppm to about 800 ppm, about 150 ppm to about 600 ppm, or about 200 ppm to about 500 ppm. Preferred concentrations include, e.g., about 30 ppm, 50 ppm, 75 ppm, 100 ppm, 125 ppm, 200 ppm, 250 ppm, 500 ppm, 750 ppm, or about 1000 ppm. CO can be administered to the patient intermittently or continuously, for 1 hour, 2, hours, 3 hours, 4 hours, 6, hours, 12 hours, or about 1, 2, 4, 6, 8, 10, 12, 14, 18, or 20 days, or greater than 20 days, before the procedure. It can be administered in the time period immediately prior to the surgery and optionally continue through the procedure, or the administration can cease at least 15 minutes before the surgery begins (e.g., at least 30 minutes, 1 hour, 2 hours 3 hours, 6 hours, or 24 hours before the surgery begins. Alternatively or in addition, CO can be administered to the patient during the procedure, e.g., by inhalation and/or topical administration. Alternatively or in addition, CO can be administered to the patient after the procedure, e.g., starting immediately after completion of the procedure, and continuing for about 1, 2, 3, 5, 7, or 10 hours, or about 1, 2, 5, 8, 10, 20, 30, 50, or 60 days, 1 year, indefinitely, or until the patient no longer suffers from, or is at risk for, the condition or disease after the completion of the procedure.

In the context of transplantation, the present invention further contemplates that other procedures known in the art for enhancing graft survival/function can be used along with the methods described herein. Such procedures include, but are not limited to immunosuppressive therapies and donor specific transfusions (DSTs). For example, a DST can be administered to a recipient prior to, during and/or after the administration of CO, HO-1, other heme-associated products, and/or NO to a recipient. Such administration, e.g., administration of DST(s) along with a treatment described herein, can be carried out prior to, during, and/or after transplantation.

Treatment of Patients with Pharmaceutical Compositions of the Present Invention

A patient can be treated with pharmaceutical compositions described herein by any method known in the art of administering liquids, solids, and/or gases to a patient.

Systemic Delivery of Pharmaceutical Compositions

Liquid and Solid Pharmaceutical Compositions

The present invention contemplates that aqueous pharmaceutical compositions can be created for systemic delivery to a patient by injection into the body, e.g., intravenously, intra-arterially, intraperitoneally, and/or subcutaneously. Liquid pharmaceutical compositions can also be prepared for oral delivery, e.g., in encapsulated or unencapsulated form, to be absorbed in any portion of the gastrointestinal tract, e.g., the stomach or small intestine. Similarly, solid pharmaceutical compositions can be created for systemic delivery to a patient, e.g., in the form of a powder or an ingestible capsule.

Liquid and solid pharmaceutical compositions typically include the active ingredient and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral and/or rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. It should be stable under the conditions of manufacture and storage, and should be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, e.g., sugars, polyalcohols such as manitol or sorbitol, or sodium chloride can be included in the composition. Prolonged absorption of the injectable compositions can be brought about by including in the composition an agent which delays absorption, for example, aluminum monostearate and gelatin. Microbeads, microspheres, or any other physiologically-acceptable methods, e.g., encapsulation, can be used to delay release or absorption of the active ingredients.

Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions, which can be aqueous or solid, generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents and/or adjuvant materials can be included as part of the composition. Tablets, pills, capsules, troches and the like can contain any of the following ingredients, and/or compounds of a similar nature: a binder such as microcrystalline cellulose, gum tragacanth or gelatin; an excipient such as starch or lactose; a disintegrating agent such as alginic acid, Primogel™, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives for transmucosal administration. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The active ingredients can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, which can be expressed as the ratio LD50/ED50.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Gaseous Pharmaceutical Compositions

Gaseous pharmaceutical compositions, e.g., pharmaceutical compositions containing NO and/or CO, can be delivered systemically to a patient by inhalation through the mouth or nasal passages to the lungs. The following methods and apparatus for administering CO compositions are illustrative of useful systemic delivery methods for the gaseous pharmaceutical compositions described herein.

Ventilators

Medical grade carbon monoxide (concentrations can vary) can be purchased mixed with air or another oxygen-containing gas in a standard tank of compressed gas (e.g., 21% O₂, 79% N₂). It is non-reactive, and the concentrations that are required for the methods of the present invention are well below the combustible range (10% in air). In a hospital setting, the gas presumably will be delivered to the bedside where it will be mixed with oxygen or house air in a blender to a desired concentration in ppm (parts per million). The patient will inhale the gas mixture through a ventilator, which will be set to a flow rate based on patient comfort and needs. This is determined by pulmonary graphics (i.e., respiratory rate, tidal volumes etc.). Fail-safe mechanism(s) to prevent the patient from unnecessarily receiving greater than desired amounts of carbon monoxide can be designed into the delivery system. The patient's carbon monoxide level can be monitored by studying (1) carboxyhemoglobin (COHb), which can be measured in venous blood, and (2) exhaled carbon monoxide collected from a side port of the ventilator. Carbon monoxide exposure can be adjusted based upon the patient's health status and on the basis of the markers. If necessary, carbon monoxide can be washed out of the patient by switching to 100% O₂ inhalation. Carbon monoxide is not metabolized; thus, whatever is inhaled will ultimately be exhaled except for a very small percentage that is converted to CO₂. Carbon monoxide can also be mixed with any level of O₂ to provide therapeutic delivery of carbon monoxide without consequential hypoxic conditions.

Face Mask and Tent

A carbon monoxide containing gas mixture is prepared as above to allow passive inhalation by the patient using a facemask or tent. The concentration inhaled can be changed and can be washed out by simply switching over to 100% O₂. Monitoring of carbon monoxide levels would occur at or near the mask or tent with a fail-safe mechanism that would prevent too high of a concentration of carbon monoxide from being inhaled.

Portable Inhaler

Compressed carbon monoxide can be packaged into a portable inhaler device and inhaled in a metered dose, for example, to permit intermittent treatment of a recipient who is not in a hospital setting. Different concentrations of carbon monoxide could be packaged in the containers. The device could be as simple as a small tank (e.g., under 5 kg) of appropriately diluted CO with an on-off valve and a tube from which the patient takes a whiff of CO according to a standard regimen or as needed.

Intravenous Artificial Lung

An artificial lung (a catheter device for gas exchange in the blood) designed for O₂ delivery and CO₂ removal can be used for carbon monoxide delivery. The catheter, when implanted, resides in one of the large veins and would be able to deliver carbon monoxide at given concentrations either for systemic delivery or at a local site. The delivery can be a local delivery of a high concentration of carbon monoxide for a short period of time at the site of the procedure, e.g., in proximity to the small intestine (this high concentration would rapidly be diluted out in the bloodstream), or a relatively longer exposure to a lower concentration of carbon monoxide (see, e.g., Hattler et al., Artif. Organs 18(11):806-812, 1994; and Golob et al., ASAIO J., 47(5):432-437, 2001).

Normobaric Chamber

In certain instances, it would be desirable to expose the whole patient to carbon monoxide. The patient would be inside an airtight chamber that would be flooded with carbon monoxide (at a level that does not endanger the patient, or at a level that poses an acceptable risk, or for non-human donors or brain-dead donors, at any desired level) without the risk of bystanders being exposed. Upon completion of the exposure, the chamber could be flushed with air (e.g., 21% O₂, 79% N₂) and samples could be analyzed by carbon monoxide analyzers to ensure no carbon monoxide remains before allowing the patient to exit the exposure system.

Topical Delivery of Pharmaceutical Compositions

Alternatively or in addition, pharmaceutical compositions can be applied directly to an organ, tissue, or area of the patient's body to be treated.

Liquid and Solid Pharmaceutical Compositions

Aqueous and solid pharmaceutical compositions can also be directly applied to an organ of a patient, or to an area of the patient targeted for treatment, by any method known in the art for administering liquids or solids to patients. For example, an aqueous or solid composition can be administered orally, e.g., by causing the patient to ingest an encapsulated or unencapsulated dose of the aqueous or solid pharmaceutical composition, to treat the interior of the gastrointestinal tract or any portion thereof. Further, liquids, e.g., saline solutions, are often injected into the gastrointestinal tract and the abdominal cavity of patients during endoscopic and laparoscopic procedures, respectively. The skilled practitioner will appreciate that similar procedures could be used to administer aqueous pharmaceutical compositions directly to an organ, tissue or cells, e.g., in the vicinity of an organ, tissue or cells to be treated, to thereby expose the organ, tissue or cells in situ to an aqueous pharmaceutical composition.

In the context of transplantation, in situ exposures can be performed by any method known in the art, e.g., by in situ flushing of the organ, tissue or cells with a liquid pharmaceutical composition prior to removal from the donor (see Oxford Textbook of Surgery, Morris and Malt, Eds., Oxford University Press (1994)). Such exposures are described in further detail below.

Gaseous Pharmaceutical Compositions

A gaseous pharmaceutical composition can be directly applied to an organ, tissue or cells of a patient, or to an area of the patient targeted for treatment, by any method known in the art for insufflating gases into a patient. For example, gases, e.g., carbon dioxide, are often insufflated into the gastrointestinal tract and the abdominal cavity of patients to facilitate examination during endoscopic and laparoscopic procedures, respectively (see, e.g., Oxford Textbook of Surgery, Morris and Malt, Eds., Oxford University Press (1994)). The skilled practitioner will appreciate that similar procedures could be used to administer gaseous pharmaceutical compositions directly to the interior of the gastrointestinal tract, or any portion thereof. Further, the skilled practitioner will appreciate that gaseous pharmaceutical compositions can be insufflated into the abdominal cavity of patients, e.g., in the vicinity of an organ to be treated, to thereby expose the organ in situ to a gaseous pharmaceutical composition.

### Surgical Procedures: Transplantation

The present invention contemplates the use of the methods described herein to treat patients who undergo transplantation. The methods can be used to treat donors, recipients and/or the organ at any step of the organ harvesting, storage, and transplant process. For example, an organ may be harvested from a donor, treated with a pharmaceutical composition ex vivo in accordance with the present invention, and transplanted into a recipient. Alternatively or in addition, the organ can be treated in situ, while still in the donor (by treatment of the donor or by treating the organ). Optionally, a pharmaceutical composition can be administered to the recipient prior to, during, and/or after the surgery, e.g., after the organ is reperfused with the recipient's blood. The composition may be administered to the donor prior to or during the process of harvesting the organ from the donor.

The term “transplantation” is used throughout the specification as a general term to describe the process of transferring an organ, tissue or cells to a patient. The term “transplantation” is defined in the art as the transfer of living organ, tissue or cells from a donor to a recipient, with the intention of maintaining the functional integrity of the transplanted organ, tissue or cells in the recipient (see, e.g., The Merck Manual, Berkow, Fletcher, and Beers, Eds., Merck Research Laboratories, Rahway, N.J., 1992). The term includes all categories of transplants known in the art. Transplants are categorized by site and genetic relationship between donor and recipient. The term includes, e.g., autotransplantation (removal and transfer of cells or tissue from one location on a patient to the same or another location on the same patient), allotransplantation (transplantation between members of the same species), and xenotransplantation (transplantations between members of different species).

The term “donor” as used herein refers to an animal (human or non-human) from whom an organ, tissue or cells can be obtained for the purposes of storage and/or transplantation to a recipient patient. The term “recipient” refers to an animal (human or non-human) into which an organ, tissue or cells is transferred.

The terms “organ rejection”, “transplant rejection” or “rejection” are art-recognized, and are used throughout the specification as a general term to describe the process of rejection of an organ, tissues, or cells in a recipient. Included within the definition are, for example, three main patterns of rejection that are usually identified in clinical practice: hyperacute rejection, acute rejection, and chronic rejection (see, e.g., Oxford Textbook of Surgery, Morris and Malt, Eds., Oxford University Press (1994)).

The term “organ(s)” is used throughout the specification as a general term to describe any anatomical part or member having a specific function in the animal. Further included within the meaning of this term are substantial portions of organs, e.g., cohesive tissues obtained from an organ. Also included within the meaning of this term are portions of an organ as small as one cell of the organ. Such organs include but are not limited to kidney, liver, heart, intestine, e.g., large or small intestine, pancreas, limbs and lungs. Also included in this definition are bones, skin, neural cells, pancreatic islets, and blood vessels.

Ex vivo exposure of an organ, tissue or cells to a pharmaceutical composition can occur by exposing the organ, tissue or cells to an atmosphere comprising a gaseous pharmaceutical composition, to a liquid pharmaceutical composition, e.g., a liquid perfusate, storage solution, or wash solution containing the pharmaceutical composition, or to both.

For example, in the context of exposing an organ, tissue or cells to a gaseous pharmaceutical composition comprising NO and/or CO, the exposure can be performed in any chamber or area suitable for creating an atmosphere that includes appropriate levels of the gases. Such chambers include, for example, incubators and chambers built for the purpose of accommodating an organ in a preservation solution. An appropriate chamber may be a chamber wherein only the gases fed into the chamber are present in the internal atmosphere, such that the concentration of CO and/or NO can be established and maintained at a given concentration and purity, e.g., where the chamber is airtight. For example, a CO₂ incubator may be used to expose an organ to a CO and/or NO composition, wherein CO or NO gas is supplied in a continuous flow from a vessel that contains the gas.

As another example, in the context of exposing an organ to an aqueous pharmaceutical composition, the exposure can be performed in any chamber or space having sufficient volume for submerging the organ, completely or partially, in an aqueous pharmaceutical composition. As yet another example, the organ may be exposed by placing the organ in any suitable container, and causing a liquid pharmaceutical composition to “wash over” the organ, such that the organ is exposed to a continuous flow of the composition.

As another option, the organ or tissue may be perfused with an aqueous pharmaceutical composition. The term “perfusion” is an art recognized term, and relates to the passage of a liquid, e.g., an aqueous pharmaceutical composition, through the blood vessels of the organ. Methods for perfusing organs ex vivo and in situ are well known in the art. An organ or tissue can be perfused with an aqueous pharmaceutical composition ex vivo, for example, by continuous hypothermic machine perfusion (see Oxford Textbook of Surgery, Morris and Malt, Eds., Oxford University Press (1994)). Optionally, in in situ or ex vivo perfusions, the organ can first be perfused with a wash solution, e.g., UW solution, to remove the donor's blood from the organ prior to perfusion with the aqueous pharmaceutical composition. Such a process could be advantageous, for example, when using pharmaceutical compositions comprising CO and/or NO to avoid inactivation by the donor's hemoglobin. As another option, the wash solution itself can be a pharmaceutical composition, e.g., a pharmaceutical composition comprising CO or NO.

As yet another example, in the context of pharmaceutical compositions comprising CO or NO, the organ may be placed, e.g., submerged, in a medium or solution that does not include CO or NO, and placed in a chamber such that the medium or solution can be made into a CO or NO composition via exposure to a CO- or NO-containing atmosphere as described herein. As still another example, the organ may be submerged in a liquid, and CO or NO may be “bubbled” into the liquid.

An organ can be harvested from a donor, and transplanted by any methods known to those of skill in the art (see, for example, Oxford Textbook of Surgery, Morris and Malt, Eds., Oxford University Press (1994)). The skilled practitioner will recognize that methods for transplanting and/or harvesting organs for transplantation may vary depending upon many circumstances, such as the age of the donor/recipient.

The present invention contemplates that any or all of the above methods for exposing an organ to a pharmaceutical composition, e.g., washing, submerging, or perfusing, can be used in a given procedure, e.g., used in a single transplantation procedure.

### Surgical Procedures Balloon Angioplasty and Surgically-Induced Intimal Hyperplasia

The methods described herein may be used to treat patients and/or a blood vessel subjected to angioplasty, bypass surgery, transplant, or any other procedure (e.g., vascular surgery) that may/will result in intimal hyperplasia and/or arteriosclerosis. Intimal hyperplasia from vascular injury subsequent to procedures such as angioplasty, bypass surgery or organ transplantation continues to limit the success of these therapeutic interventions. The term “intimal hyperplasia” is an art-recognized term and is used herein to refer to proliferation of cells, e.g., smooth muscle cells, within the intima of a blood vessel. The skilled practitioner will appreciate that intimal hyperplasia can be caused by any number of factors, e.g., mechanical, chemical and/or immunological damage to the intima. Intimal hyperplasia can often be observed in patients, for example, following balloon angioplasty or vascular surgery, e.g., vascular surgery involving vein grafts (e.g., transplant surgery). The term “angioplasty” is an art-recognized term and refers to any procedure, singly or in combination, involving remodeling of a blood vessel, e.g., dilating a stenotic region in a patient's vasculature to restore adequate blood flow beyond the stenosis. Such procedures include percutaneous transluminal angioplasty (PTA), which employs a catheter having an expansible distal end, i.e., an inflatable balloon (known as “balloon angioplasty”); laser angioplasty; extraction atherectomy; directional atherectomy; rotational atherectomy; stenting; and any other procedure for remodeling a blood vessel, e.g., an artery. “Arteriosclerosis,” “arteriosclerotic lesion,” “arteriosclerotic plaque,” and “arteriosclerotic condition” are also art recognized term terms, and are used herein to describe a thickening and hardening of the arterial wall. The term “vasculature” as used herein refers to the vascular system (or any part thereof) of a body, human or non-human, and includes blood vessels, e.g., arteries, arterioles, veins, venules, and capillaries. The term “restenosis” refers to re-narrowing of an artery following angioplasty.

Individuals considered at risk for developing intimal hyperplasia or arteriosclerosis may benefit particularly from the invention, primarily because prophylactic CO treatment can be administered before a procedure is performed on a patient or before there is any evidence of intimal hyperplasia or an arteriosclerotic plaque. Individuals “at risk” include, e.g., patients that have or will have any type of mechanical, chemical and/or immunological damage to the intima, e.g., patients that will or have undergone surgery, e.g., transplant surgery, and/or angioplasty. Skilled practitioners will appreciate that a patient can be determined to be at risk for intimal hyperplasia or arteriosclerosis by any method known in the art, e.g., by a physician's diagnosis.

A patient can be treated according to the methods of the present invention before, during and/or after the surgical procedure or angioplasty. Further, if desired, blood vessels can be exposed to the pharmaceutical compositions described herein in situ and/or ex vivo, as described above in the context of organ transplants. The vessel may be exposed to a gaseous pharmaceutical composition, and/or to a liquid pharmaceutical composition, e.g., a liquid perfusate, storage solution, or wash solution having the active ingredient(s) dissolved therein.

### Disorders and Conditions

The methods of the present invention can be used to treat one or more of the following inflammatory, respiratory, cardiovascular, renal, hepatobiliary, reproductive or gastrointestinal disorders; shock; or cellular proliferative and/or differentiative disorders; and to reduce the effects of ischemia; and to aid in wound healing.

Respiratory Disorders

Examples of respiratory conditions include, but are not limited to asthma; Acute Respiratory Distress Syndrome (ARDS), e.g., ARDS caused by peritonitis, pneumonia (bacterial or viral), or trauma; idiopathic pulmonary diseases; interstitial lung diseases, e.g., Interstitial Pulmonary Fibrosis (IPF); pulmonary emboli; Chronic Obstructive Pulmonary Disease (COPD); emphysema; bronchitis; cystic fibrosis; lung cancer of any type; lung injury, e.g., hyperoxic lung injury; Primary Pulmonary Hypertension (PPH); secondary pulmonary hypertension; and sleep-related respiratory disorders, e.g., sleep apnea.

Cardiovascular Disorders

Cardiovascular disorders include disorders involving the cardiovascular system, e.g., the heart, the blood vessels, and/or the blood. A cardiovascular disorder can be caused, for example, by an imbalance in arterial pressure, a malfunction of the heart, or an occlusion of a blood vessel, e.g., by a thrombus. Examples of such disorders include congestive heart failure, peripheral vascular disease, pulmonary vascular thrombotic diseases such as pulmonary embolism, stroke, ischemia-reperfusion (I/R) injury to the heart, atherosclerosis, and heart attacks.

Renal Disorders

Disorders involving the kidney include but are not limited to pathologies of glomerular injury such as in situ immune complex deposition and cell-mediated immunity in glomerulonephritis, damage caused by activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, e.g., poststreptococcal glomerulonephritis and nonstreptococcal acute glomerulonephritis, rapidly progressive glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis) and chronic glomerulonephritis. Disorders of the kidney also include infections of the genitourinary tract.

Hepatobiliary Disorders

Disorders involving the liver include but are not limited hepatitis, cirrhosis and infectious disorders. Causative agents of hepatitis include, for example, infections, e.g., infection with specific hepatitis viruses, e.g., hepatitis A, B, C, D, E, and G viruses; or hepatotoxic agents, e.g., hepatotoxic drugs (e.g., isoniazid, methyldopa, acetaminophen, amiodarone, and nitrofurantoin), and toxins (e.g., endotoxin or environmental toxins). Hepatitis may occur postoperatively in liver transplantation patients. Further examples of drugs and toxins that may cause hepatitis (i.e., hepatotoxic agents) are described in Feldman: Sleisenger & Fordtran's Gastrointestinal and Liver Disease, 7th ed., Chapter 17 (Liver Disease Caused by Drugs, Anesthetics, and Toxins), the contents of which are expressly incorporated herein by reference in their entirety. Such examples include, but are not limited to, methyldopa and phenyloin, barbiturates, e.g., phenobarbital; sulfonamides (e.g., in combination drugs such as co-trimoxazole (sulfamethoxazole and trimethoprim); sulfasalazine; salicylates; disulfuram; β-adrenergic blocking agents e.g., acebutolol, labetalol, and metoprolol); calcium channel blockers, e.g., nifedipine, verapamil, and diltiazem; synthetic retinoids, e.g., etretinate; gastric acid suppression drugs e.g., oxmetidine, ebrotidine, cimetidine, ranitidine, omeprazole and famotidine; leukotriene receptor antagonists, e.g., zafirlukast; anti-tuberculosis drugs, e.g., rifampicin and pyrazinamide; antifungal agents, e.g., ketoconazole, terbinafine, fluconazole, and itraconazole; antidiabetic drugs, e.g., thiazolidinediones, e.g., troglitazone and rosiglitazone; drugs used in neurologic disorders, e.g., neuroleptic agents, antidepressants (e.g., fluoxetine, paroxetine, venlafaxine, trazodone, tolcapone, and nefazodone), hypnotics (e.g., alpidem, zolpidem, and bentazepam), and other drugs, e.g., tacrine, dantrolene, riluzole, tizanidine, and alverine; nonsteroidal anti-inflammatory drugs, e.g., bromfenac; COX-2 inhibitors; cyproterone acetate; leflunomide; antiviral agents, e.g., fialuridine, didanosine, zalcitabine, stavudine, lamivudine, zidovudine, abacavir; anticancer drugs, e.g., tamoxifen and methotrexate; recreational drugs, e.g., cocaine, phencyclidine, and 5-methoxy-3,4-methylenedioxymethamphetamine; L-asparaginase; amodiaquine; hycanthone; anesthetic agents; e.g., halothane, enflurane, and isoflurane; vitamins e.g., vitamin A; and dietary and/or environmental toxins, e.g., pyrrolizidine alkaloids, toxin from Amanita phalloides or other toxic mushrooms, aflatoxin, arsenic, Bordeaux mixture (copper salts and lime), vinyl chloride monomer; carbon tetrachloride, beryllium, dimethylformamide, dimethylnitrosamine, methylenedianiline, phosphorus, chlordecone (Kepone), 2,3,7,8-tetrachloro-dibenzo p-dioxin (TCDD), tetrachloroethane, tetrachloroethylene, 2,4,5-trinitrotoluene, 1,1,1-trichloroethane, toluene, and xylene, and known “herbal remedies,” e.g., ephedrine and eugenol.

Symptoms of hepatitis can include fatigue, loss of appetite, stomach discomfort, and/or jaundice (yellowing of the skin and/or eyes). More detailed descriptions of hepatitis are provided, for example, in The Merck Manual of Diagnosis and Therapy, 17^{th} Edition, Section 4, Chapter 42, Section 4, Chapter 44, and Section 4, Chapter 40, the contents of which are expressly incorporated herein by reference in their entirety.

Skilled practitioners will appreciate that a patient can be diagnosed by a physician as suffering from hepatitis by any method known in the art, e.g., by assessing liver function, e.g., using blood tests for serum alanine aminotransferase (ALT) levels, alkaline phosphatase (AP), or bilirubin levels.

Individuals considered at risk for developing hepatitis may benefit particularly from the invention, primarily because prophylactic treatment can begin before there is any evidence of hepatitis. Individuals “at risk” include, e.g., patients infected with hepatitis viruses, or individuals suffering from any of the conditions or having the risk factors described herein (e.g., patients exposed to hepatotoxic agents, alcoholics). The skilled practitioner will appreciate that a patient can be determined to be at risk for hepatitis by a physician's diagnosis.

Gastrointestinal Disorders

Gastrointestinal disorders include but are not limited to ileus (of any portion of the gastrointestinal tract, e.g., the large or small intestine), inflammatory bowel disease, e.g., specific inflammatory bowel disease, e.g., infective specific inflammatory bowel disease, e.g., amoebic or bacillary dysentery, schistosomiasis, campylobacter enterocolitis, yersinia enterocolitis, or enterobius vermicularis; non-infective specific inflammatory bowel disease, e.g., radiation enterocolitis, ischaemic colitis, or eosinophilic gastroenteritis; and non-specific bowel disease, e.g., ulcerative colitis, indeterminate colitis, and Crohn's disease; necrotizing enterocolitis (NEC), and pancreatitis.

Cellular Proliferative and/or Differentiative Disorders and Angiogenesis

Examples of cellular proliferative and/or differentiative disorders include, but are not limited to, carcinoma, sarcoma, metastatic disorders, and hematopoietic neoplastic disorders, e.g., leukemias. A metastatic tumor can arise from a multitude of primary tumor types, including but not limited to those of prostate, colon, lung, breast and liver origin.

The term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” is any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

Cancers that may be treated using the methods and compositions of the present invention include, for example, cancers of the stomach, colon, rectum, mouth/pharynx, esophagus, larynx, liver, pancreas, lung, breast, cervix uteri, corpus uteri, ovary, prostate, testis, bladder, skin, kidney, brain/central nervous system, head, neck and throat; Hodgkins disease, non-Hodgkins leukemia, sarcomas, choriocarcinoma, and lymphoma, among others.

The methods of the present invention can also be used to inhibit unwanted (e.g., detrimental) angiogenesis in a patient and to treat angiogenesis dependent/associated conditions associated therewith. As used herein, the term “angiogenesis” means the generation of new blood vessels in a tissue or organ. An “angiogenesis dependent/associated condition” includes any process or condition that is dependent upon or associated with angiogenesis. The term includes conditions that involve cancer, as well as those that do not. Angiogenesis dependent/associated conditions can be associated with (e.g., arise from) unwanted angiogenesis, as well as with wanted (e.g., beneficial) angiogenesis. The term includes, e.g., solid tumors; tumor metastasis; benign tumors, e.g., hemangiomas, acoustic neuromas, neurofibromas, trachomas, and pyogenic granulomas; rheumatoid arthritis, lupus, and other connective tissue disorders; psoriasis; rosacea; ocular angiogenic diseases, e.g., diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation. Other processes in which angiogenesis is involved include reproduction and wound healing. Because of its anti-VEGF properties, CO can also be useful in the treatment of diseases of excessive or abnormal stimulation of endothelial cells. Such diseases include, e.g., intestinal adhesions, atherosclerosis, scleroderma, and hypertrophic scars, e.g., keloids, as well as endothelial cell cancers that are sensitive to VEGF stimulation.

Individuals considered at risk for developing cancer may benefit particularly from the invention, primarily because prophylactic treatment can begin before there is any evidence of the disorder. Individuals “at risk” include, e.g., individuals exposed to carcinogens, e.g., by consumption, e.g., by inhalation and/or ingestion, at levels that have been shown statistically to promote cancer in susceptible individuals. Also included are individuals at risk due to exposure to ultraviolet radiation, or their environment, occupation, and/or heredity, as well as those who show signs of a precancerous condition such as polyps. Similarly, individuals in very early stages of cancer or development of metastases (i.e., only one or a few aberrant cells are present in the individual's body or at a particular site in an individual's tissue)) may benefit from such prophylactic treatment. The skilled practitioner will appreciate that a patient can be determined to be at risk for cancer by any method known in the art, e.g., by a physician's diagnosis. Skilled practitioners will also appreciate that chemotherapy, radiation therapy, immunotherapy, gene therapy, and/or surgery can be administered in combination with the treatments described herein, for example, to treat cancer.

Neurological Disorders

The methods of the present invention can also be used to treat neurological disorders. Neurological disorders include, but are not limited to disorders involving the brain, e.g., degenerative diseases affecting the cerebral cortex, including Alzheimer's disease, and degenerative diseases of basal ganglia and brain stem, including Parkinsonism and idiopathic Parkinson's disease (paralysis agitans). Further, the methods may be used to treat pain disorders. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York: McGraw-Hill); pain associated with musculoskeletal disorders, e.g., joint pain; tooth pain; headaches; pain associated with surgery; pain related to irritable bowel syndrome; or chest pain. Also included in this category are seizure disorders, e.g., epilepsy.

Inflammatory Disorders

The methods of the present invention can be used to treat inflammatory disorders. The terms “inflammatory disorder(s)” and “inflammation” are used to describe the fundamental pathological process consisting of a dynamic complex of reactions (which can be recognized based on cytologic and histologic studies) that occur in the affected blood vessels and adjacent tissues in response to an injury or abnormal stimulation caused by a physical, chemical or biologic agent, including the local reactions and resulting morphologic changes, the destruction or removal of the injurious material, and the responses that lead to repair and healing. Inflammation is characterized in some instances by the infiltration of immune cells (e.g., monocytes/macrophages, natural killer cells, lymphocytes (e.g., B and T lymphocytes)). In addition, inflamed tissue may contain cytokines and chemokines that are produced by the cells that have infiltrated into the area. Often, inflammation is accompanied by thrombosis, including both coagulation and platelet aggregation. The term inflammation includes various types of inflammation such as acute, chronic, allergic (including conditions involving mast cells), alternative (degenerative), atrophic, catarrhal (most frequently in the respiratory tract), croupous, fibrinopurulent, fibrinous, immune, hyperplastic or proliferative, subacute, serous and serofibrinous inflammation. Inflammation localized in the gastrointestinal tract, or any portion thereof, liver, heart, skin, spleen, brain, kidney, pulmonary tract, and the lungs can be treated with the methods of the present invention. Inflammation associated with shock, e.g., septic shock, hemorrhagic shock caused by any type of trauma, and anaphylactic shock can also be treated. Further, it is contemplated that the methods of the present invention could be used to treat rheumatoid arthritis, lupus, and other inflammatory and/or autoimmune diseases; heightened inflammatory states due to immunodeficiency, e.g., due to infection with HIV; and hypersensitivities.

Wound Healing

Based on the anti-inflammatory properties of HO-1 and heme degradation products, the present invention contemplates that the methods described herein can be used to promote wound healing (e.g., in transplanted, lacerated (e.g., due to surgery), or burned skin). They would typically be applied locally to the wound (e.g., as a wound dressing, lotion, or ointment), but could be delivered systemically as well.

Reproductive Disorders

The methods described herein can be used to treat or prevent certain reproductive disorders, e.g., impotence and/or inflammation associated with sexually transmitted diseases. Further, the methods of the present invention can be used to prevent premature uterine contractions, and may be used to prevent premature deliveries and menstrual cramps.

### EXAMPLES

A number of embodiments of the invention have been described. Nevertheless, it will be understood that various modifications may be made without departing from the spirit and scope of the invention. Accordingly, other embodiments are within the scope of the following claims.

### Interrelationship Between Co/Ho-1 and NO/iNOS in Providing Protection Against Acute Liver Failure

### Animals

Male C57BL/6J (Charles Rivers Laboratories, Bar Harbor, Me.), 8-12-wk-old inos^{-/-} mice and wild type littermates (bred/maintained at the University of Pittsburgh) were used for in vivo experiments.

### Acute Hepatic Injury Models

Groups of mice were administered TNF-α/D-gal (0.3 μg/8 mg/mouse, i.p., respectively). Depending on the experimental condition, some mice received CO (250 ppm), the selective NO donor O₂-vinyl 1-(pyrrolidin-1-yl) diazen-1-ium-1,2-diolate (V-PYRRO; 10 mg/kg subcutaneously (s.c.), Alexis Biochem., San Diego, Calif.) or cobalt protoporphyrin (CoPP, 5 mg/kg, intraperitoneally (i.p.), Frontier Scientific, Logan, Utah). Additionally, the selective inhibitor of iNOS L-N6-(1-iminoethyl)-lysine-dihydrochloride (L-NIL; 5 mg/kg, i.p., Alexis Biochemicals) or the HO-1 inhibitor tin protoporphyrin (SnPP; 50 μmol/kg, i.p., Frontier Scientific) was administered when specified.

### Hepatocyte Cell Culture.

Mouse primary hepatocytes were harvested from C57BL/6J, mkk3^{-/-}, inos^{-/-} (in-house breeding colony), or hmox-1^{-/-} mice as described in Kim et al. (J. Biol. Chem. 272: 1402-1411 (1997)). Hepatocytes were used on days 1-3 following harvest.

### Induction of Hepatocyte Death/Apoptosis

Cells were treated with TNF-α (10 ng/ml) and actinomycin-D (Act-D; 200 ng/ml, Sigma Chemical Co. St. Louis, Mo.) to induce cell death. TNF-α/ActD treatment has been demonstrated to induce cell death, specifically apoptosis, in primary hepatocytes (see, e.g., Kim et al. (J. Biol. Chem. 272: 1402-1411 (1997)). Hepatocytes were treated with CO, the NO donor s-nitroso-N-acetyl-penicillamine (SNAP; 250-750 μM), and/or additional pharmacologic agents where indicated. Twelve hours after TNF-α/ActD treatment, cells were washed and stained with crystal violet to determine viability as previously described (Id.). Where indicated, the selective in vitro inhibitor of iNOS, L-N5-(1-iminoethyl)-ornithine-2HCl (LNIO; 1-2 mM; Calbiochem, San Diego, Calif.) was administered.

### Immunoblot Analysis

Western blot analysis was performed on primary hepatocytes in culture or from liver homogenates with antibodies to iNOS (Transduction Laboratories, Lexington, Ky.; 1:1000), HO-1 (Calbiochem; 1:2000), or β-actin (Sigma Chemical; 1:5000). Thirty μg protein in cell culture experiments or 100 μg protein from liver homogenates was loaded per well for SDS-PAGE.

### Serum Alanine Aminotransferase Levels

Serum alanine aminotransferase (ALT) levels in mice were measured using a test kit in accordance with the manufacturer's instructions (Sigma, St. Louis Mo.).

### CO Exposure

The animals were exposed to CO at a concentration of 250 ppm. Briefly, 1% CO in air was mixed with air (21% oxygen) in a stainless steel mixing cylinder and then directed into a 3.70 ft³ glass exposure chamber at a flow rate of 12 L/min. A CO analyzer (Interscan, Chatsworth, Calif.) was used to measure CO levels continuously in the chamber. CO concentrations were maintained at 250 ppm at all times. Animals were placed in the exposure chamber as required.

### The Role of HO-1 in CO Protection Against Acute Liver Failure

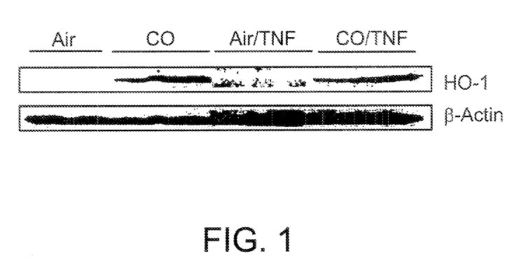

Whether CO and NO exert protection against acute liver failure through an HO-1-dependent mechanism was investigated. The data are presented in FIGS. 1, 2, 3, and 4.

To generate the data presented in FIG. 1, immunoblotting was performed to observe HO-1 expression in the livers of mice that received TNF-α/D-gal in the presence and absence of CO (250 ppm). CO-treated mice showed a significant increase in HO-1 expression in both the presence and absence of TNF-α/D-gal.

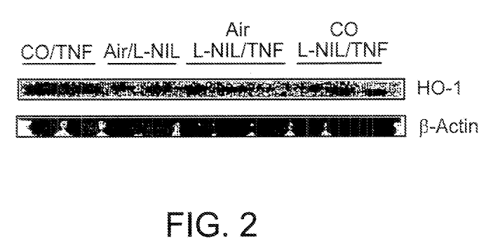

To assess the role of iNOS on TNF-α/D-gal-induced HO-1 expression in the liver (data presented in FIG. 2), mice were administered L-NIL (5 mg/kg, i.p.) 2 hr prior to pre-treatment with CO (250 ppm) and every 2 hr thereafter. Control mice received L-NIL and remained in room air. Note in FIG. 2 that CO increased HO-1 expression in vehicle-treated mice, but was unable to induce expression when iNOS was inhibited. L-NIL treatment alone had a minimal effect on HO-1 expression.

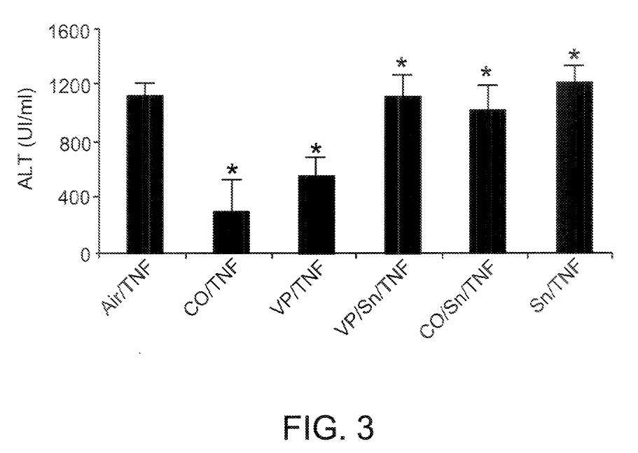

To test the protective role of CO-induced HO-1 (data presented in FIG. 3), mice were given SnPP (50 μmol/kg, s.c.), the selective inhibitor of HO-1, 5 hr prior to CO. Alternatively, the mice were given VPYRRO (VP), an NO donor (10 mg/kg, s.c.). VP was selectively designed to deliver NO directly to the liver. One hour after the initial VP dose, the animals were exposed to CO for 1 hr prior to administration of TNF-α/D-gal (see above). Serum ALT levels were determined 6-8 hr later. Note that CO was not able to provide protection in animals where HO-1 activity was blocked. VP, when administered 2 hr prior and then every 2 hr thereafter, provided protection against injury as determined 8 hour later by serum ALT measurements.

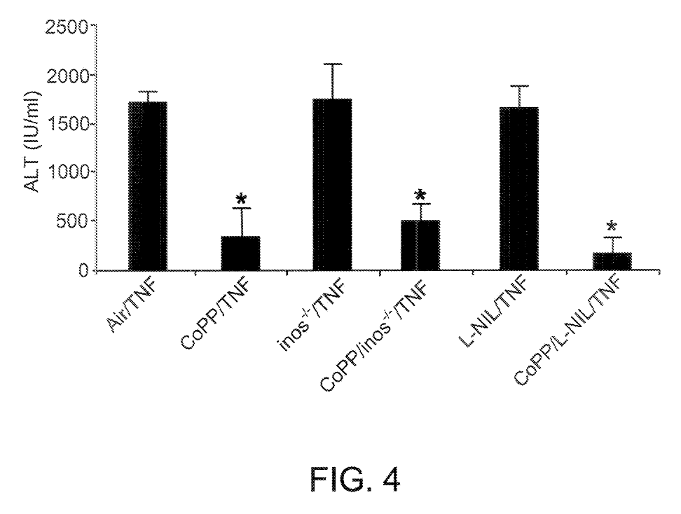

To generate the data presented in FIG. 4, wild type C57BL/6J mice were pretreated for 24 hr with L-NIL in the drinking water (4.5 mM) as described in Stenger et al. (J. Exp. Med. 183: 1501-1514 (1996)). These mice and inos^{-/-} mice were then administered CoPP. L-NIL was maintained in the water throughout the experiment. Control and inos^{-/-} mice received normal drinking water. Twenty-four hr after administration of CoPP, TNF-α/D-gal was administered and serum ALT determined 6-8 hr later. Note in FIG. 4 that induction of HO-1 provides protection regardless of the presence of iNOS.

Immunoblotting of liver extracts from mice treated with CO in the presence or absence of TNF-α/D-gal showed up-regulation of HO-1 (FIG. 1). The addition of the iNOS inhibitor L-NIL to these above groups, which abrogated the protection (FIG. 3), also prevented up-regulation of HO-1 (FIG. 2). To determine whether HO-1 was central to CO-elicited hepatoprotection, tin protoporphyrin-IX (SnPP, 50 μmol/kg, s.c., Frontier Scientific) was used as a selective inhibitor of HO-1 activity. SnPP significantly diminished the protective effects of CO in this model (FIG. 3). SnPP administration in the absence of TNF-α/D-gal had no deleterious or protective effects (data not shown). These results suggest that up-regulation of HO-1 is important to the protective effects of CO.

To determine if up-regulation of HO-1 would also be needed if protection was initiated by NO, mice were treated with the pharmacological NO donor V-PYRRO/NO. This agent is metabolized by the liver, resulting in release of NO by hepatocytes. V-PYRRO/NO also provides protection following LPS/D-gal or TNF-α/D-gal administration. Mice were randomized and treated with TNF-α/D-gal with or without SnPP to evaluate the role of HO-1. V-PYRRO/NO was protective, as assayed by serum ALT. However, SnPP abrogated the ability of this NO donor to protect against liver damage (FIG. 3). Thus, it appears that CO- or NO-initiated hepatoprotection is at least partially dependent on HO-1.

Because these data suggest that CO and NO require HO-1 activity to protect against TNF-α-induced hepatocyte death, whether protection mediated by HO-1 requires iNOS activity was investigated. Using inos^{-/-} mice, HO-1 was induced via administration of CoPP. TNF-α/D-gal was injected 24 hr thereafter, at the peak of HO-1 expression, and liver damage was assessed 6-8 hr later. The results show that induction of HO-1 was able to significantly prevent liver injury independently of iNOS activity with a >50% reduction in serum ALT (FIG. 4). These results were confirmed using L-NIL. Mice were pre-treated with drinking water containing L-NIL (4.5 mM) for 24 hours. This method effectively inhibits NOS activity. Control mice received normal water. Subsequently, CoPP was administered to induce HO-1 expression and 24 hours thereafter mice were challenged with TNF-α/D-gal. L-NIL treatment alone did not change the severity of injury induced in this model. All animals receiving CoPP (with and without L-NIL) were protected from liver injury (FIG. 4).

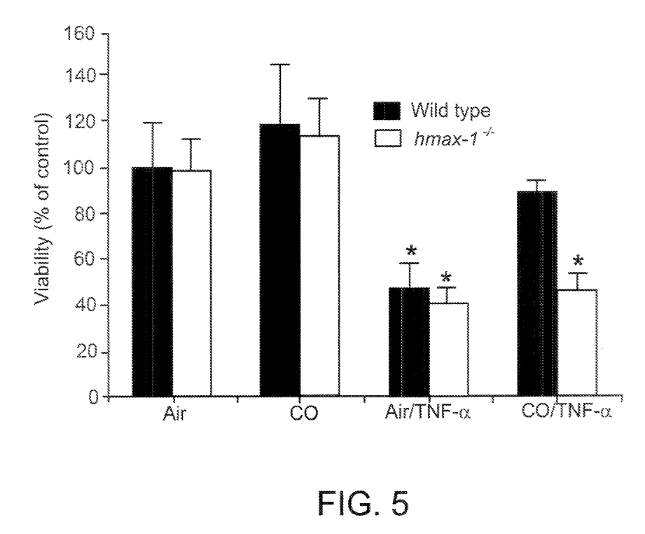

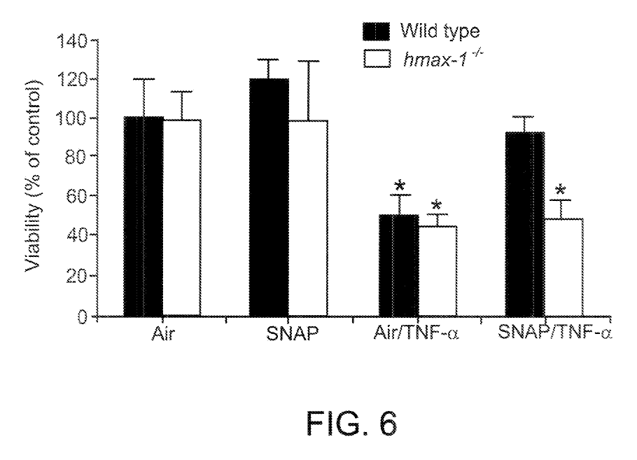

Whether HO-1 expression is required for CO- or NO-induced protection from TNF-α/ActD-induced hepatocyte cell death was investigated. The data are presented in FIGS. 5 and 6.

To generate the data presented in FIG. 5, mouse hepatocytes were isolated from HO-1 null mice (hmox-1^{-/-}) and wild type (C57BL/6J) littermates, pretreated for 1 hour with CO (250 ppm), and treated with TNF-α/ActD. Viability was assayed as described above. CO significantly protected wild type hepatocytes, but was unable to protect hepatocytes isolated from hmox-1^{-/-} mice.

To generate the data presented in FIG. 6, mouse hepatocytes were isolated from HO-1 null mice (hmox-1^{-/-}) and wild type (C57BL/6J) littermates, pretreated with the NO donor SNAP (500 μM), and then treated with TNF-α/ActD 1 hour later. SNAP has been demonstrated to protect hepatocytes in this model. SNAP significantly protected against cell death in wild type hepatocytes but did not provide significant protection against cell death in hepatocytes isolated from hmox-1^{-/-} mice. As discussed above, air-treated wild type and hmox-1^{-/-} cells exposed to TNF-α/ActD underwent cell death as expected, while CO— or NO— treated wild type cells were protected in the presence of TNF-α/ActD (FIGS. 5 and 6). The protection conferred by CO and NO was lost in cells lacking functional HO-1 (hmox-1^{-/-}). Thus, it appears that HO-1 can provide protection in this model without the involvement of iNOS, suggesting that HO-1 or one or more of its catalytic products can, in part, exert cytoprotective effects in this model.

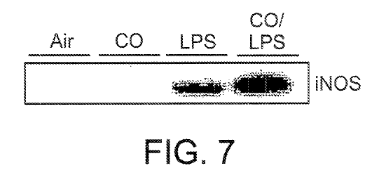

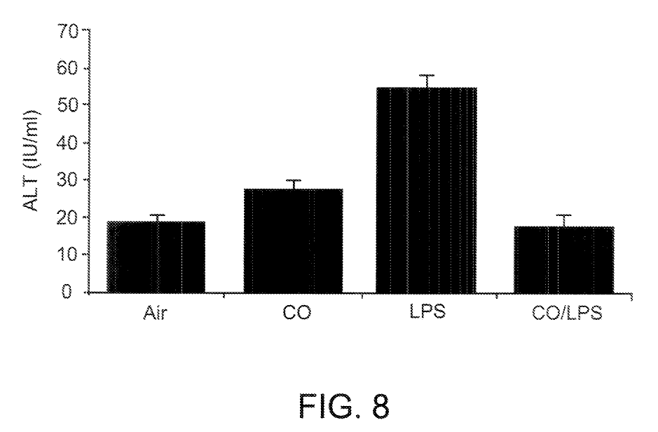

Whether CO augments LPS-induced iNOS expression in the liver of rats and whether CO can inhibit lipopolysaccharide (LPS)-induced liver injury was investigated. The data are presented in FIGS. 7 and 8. To generate the data presented in FIG. 7, rats were pretreated one hour with CO (250 ppm) and then administered LPS (50 mg/kg, i.v.). Liver samples were harvested and analyzed for iNOS expression by Western blot 8 hours later. The results show that LPS induced an increase in iNOS protein expression, which was significantly augmented in the presence of CO. These data demonstrate that CO augments LPS-induced iNOS expression in the liver of rats. To generate the data presented in FIG. 8, rats were administered 50 mg/kg, LPS, i.v.±CO (250 ppm) and blood was taken 8 hours later for serum ALT determination. ALT was measured using a test kit (Sigma, St. Louis Mo.). Data is mean ±SD of 4-6 rats/group. Correlating with the data presented in FIG. 7, these data demonstrate that CO can inhibit LPS-induced liver injury as assessed by increased serum ALT levels.

1-15. (canceled)

16. A method of transplanting an organ, a tissue, or cells, the method comprising:
(a) administering to a donor:
(i) a pharmaceutical composition comprising nitric oxide; and
(ii) a second treatment selected from the group consisting of: inducing HO-1 in the donor; expressing HO-1 in the donor; inducing apoferritin in the donor;
expressing apoferritin in the donor; and administering to the donor a pharmaceutical composition comprising HO-1, carbon monoxide, bilirubin, biliverdin, ferritin, iron, desferoxamine, salicylaldehyde isonicotinoyl hydrazone, iron dextran, or apoferritin;
(b) obtaining an organ, a tissue, or cells from the donor; and
(c) transplanting the organ, tissue, or cells into a recipient, wherein the nitric oxide and second treatment administered in step (a) are sufficient to enhance survival or function of the organ, tissue, or cells after transplantation into the recipient.

17-23. (canceled)
